(19) United States
(12) Patent Application Publication
(10) Pub. No.: US 2024/0395390 A1
(43) Pub. Date: Nov. 28, 2024

(54) SYSTEM AND METHOD FOR PERSONALIZED MOTIVATION MANAGEMENT

(71) Applicant: NAVAPRANA TECHNOLOGIES INC., SANTA CLARA, CA (US)

(72) Inventors: SANTHOSH K PURATHEPPARAMBIL, SANTA CLARA, CA (US); SWASTIK S PURATHEPPARAMBIL, SANTA CLARA, CA (US); PRANAV S PURATHEPPARAMBIL, SANTA CLARA, CA (US); ROHAN PURI, SAN JOSE, CA (US); NAVPREET BAWA, San Jose, CA (US)

(21) Appl. No.: 18/321,371

(22) Filed: May 22, 2023

(51) Int. Cl. G16H 20/70

(52) U.S. Cl. CPC G16H 20/70

(57) ABSTRACT

The embodiments herein provide a method and a system for personalized motivation management platform to improve productivity and well-being of a user. The system extracts various attributes, including the user's current motivation level, behavior, skill level, and desired goals, from an application or website that the user interacts with or from external devices such as smartwatches that track their activities. Based on these attributes, the system generates one or more motivation models for each user and calculates a motivation score. Using these models and scores, the system generates targeted control elements specific to a particular user and delivers them to the user's device through one or more channels in order to modify the user's motivation. The system provides individuals with a personalized self-improvement system and skills by using management platform's tools, methods, and knowledge, so that they can successfully achieve their desired behavior or goals.

### BACKGROUND

### Technical Field

The embodiments herein relate to the field of software applications. The embodiments herein are particularly related to a system and method for managing motivation in users. The embodiments herein are more particularly related to a method and system personalized motivation management platform to provide individuals with a personalized self-improvement system and skills by using the management platform's tools, methods, and knowledge, so that they can successfully achieve their desired behavior or goals.

### Description of the Related Art

People's goals and aspirations mostly rely on their motivation, but motivation itself is rarely managed. This is a problem because many people believe that to increase their productivity, they must work all the time with great effort. However, that leads to many people becoming burnt out, tired, losing efficiency, and giving up. Motivation is crucial for individuals and companies to achieve their goals and improve productivity and well-being. However, it is not often actively managed and individuals tend to follow their default behavior rather than striving towards their desired state. Currently, there are no systems in the market that actively manage motivation and help individuals achieve their goals. The only available options are motivational content and training on goal setting, which assume that the individual already has sufficient motivation.

Furthermore, there are life-style coaches, books and apps for habit creation, some apps for motivation improvement but the methodology is video-based training or quotes and expecting people get motivated. There is no system of measuring and managing motivation and its elements. Personal coaches are expensive and most people can't afford the trained professional. Even with the coaches not enough systematic approaches to this problem. The concept of actively managing motivation and channeling that motivation to achieve goals is not effectively being used. No systems, methods, and tools are available in the market today.

Hence, there is need for a method and a system for personalized motivation management, that helps individuals and organizations to achieve their goals by actively managing and improving individual's motivation. Still there is a need for a system and method for identifying and addressing factors that may be hindering motivation, such as a lack of energy to perform tasks or clear goals, lack of progress or achievement, or a negative mindset.

The above-mentioned shortcomings, disadvantages and problems are addressed herein, and which will be understood by reading and studying the following specification.

### OBJECTIVES OF THE EMBODIMENTS HEREIN

The principal object of the embodiments herein is to provide a method and system for personalized motivation management.

Another object of the embodiments herein is to provide a method and system for personalized motivation management by helping individuals or organizations achieve their goals by actively managing and improving individual's motivation.

Yet another object of the embodiments herein is to provide a method and a system for personalized motivation management by creating a personalized user text for a user with a third-party Artificial Intelligence/Machine Learning models using third party or open-source AI/ML libraries through an application or algorithm.

Yet another object of the embodiments herein is to provide one or more motivation models.

Yet another object of the embodiments herein is to manage motivation by calculating the motivation score of the users.

Yet another object of the embodiments herein is to provide a method and a system for personalized motivation management by using motivation management platform's tools, methods, and knowledge so that the user can successfully achieve their desired behavior or goals.

These and other objects and advantages of the present invention will become readily apparent from the following detailed description taken in conjunction with the accompanying drawings.

### SUMMARY

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The other objects and advantages of the embodiments herein will become readily apparent from the following description taken in conjunction with the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

This Summary is provided to introduce a selection of concepts in a simplified form that is further described below in the Detailed Description. This Summary is not intended to identify key or essential features of the claimed subject matter, nor is it intended to be used as an aid in determining the scope of the claimed subject matter.

The various embodiments herein provide a method and system for personalized motivation management. The method and system for personalized motivation management help to manage motivation in a plurality of users to improve the plurality of users, productivity, and well-being. Furthermore, the system for personalized motivation management also helps the plurality of users to manage their motivation and achieve their goals, thereby increasing motivation throughout the day. In addition, the system for personalized serves as the basis for the continuous collection of various data elements, including several categories of information, with numerous subcategories and factors. The system is configured to gather data using a questionnaire, applications, websites, and devices the plurality of users engage with to provide effective motivation models.

According to one embodiment herein, a method for personalized motivation management is provided. The method comprises extracting a plurality of motivation attributes from a plurality of users, through external applications, websites, and external devices the plurality of users interact with, and additionally collecting external data through an application programming interface. The plurality of motivation attributes includes the user's current motivation level, behavior, skill level, and desired goals. The method further comprises dynamically generating one or more motivation models for each of the plurality of users based on motivation modeling criteria derived from a plurality of motivation factors. The plurality of motivation factors comprises internal motivation factors and external motivation factors. The internal motivation factors include psychological needs, aspirations, mindset, advancement, cognitive skills, and energy state and the external motivation factors include environment context, rewards, and incentives. Furthermore, the method comprises generating a motivation score for each of the plurality of users by measuring the plurality of users' motivation and scoring the plurality of motivation factors and associated plurality of motivation sub-factors. The plurality of user's motivation is measured through self-assessment questions, system calculation, and user activity data. In addition, the method includes dynamically generating one or more targeted, control elements specific to a target user, among the plurality of users using one or more motivation models, the motivation score, and one or more content libraries. The one or more targeted, control elements include the plurality of motivation factors influencing the plurality of users' behavior and help in achieving the motivation goal. Finally, the method includes rendering the one or more targeted, control elements on a user device of the target user through a plurality of delivery channels. Furthermore, the user device comprises a smartwatch, smartphone, computer, and/or laptop. The plurality of delivery channels include smartwatches, smartphones, applications in smartwatches and smartphones, bots including Microsoft® Teams, and web interfaces.

According to one embodiment herein, the plurality of motivation attributes from the plurality of users is also extracted by the method through a questionnaire. Moreover, the external applications include chatbots, mobile applications, web interfaces, and/or web portals and the external devices include smart watches, smartphones, computers, and/or laptops. The chatbots give an interactive experience for the plurality of users working with the platform. The plurality of users can type in questions and the chatbot provides guidance and relevant charts/details related to motivation. The mobile applications are easy-to-use applications for the plurality of users to interact with the system to enter the data. Furthermore, the web interfaces are an alternative to mobile applications, easy for blog reading, and help to review various reports.

According to one embodiment herein, one or more motivation models is a digital twin of the plurality of users, which represents the plurality of users' motivation level. Furthermore, the method for personalized motivation management also assists the plurality of users in recognizing the user's motivation level at that moment and maintaining the motivation necessary to accomplish the plurality of users' goals. The plurality of users includes an individual user, a company, and or a lifestyle coach.

According to one embodiment herein, one or more motivation models generated by the method for personalized motivation management serve as a model of the desired future state by providing visibility and insights into the current state, and by offering recommendations for the future state. The recommendations for the future state comprise a range of knowledge content and skill-building activities for the plurality of users to achieve their goals. Furthermore, the recommendations for the future state are delivered to the plurality of users as a micro-content delivered directly through the applications or through channels including WhatsApp, slack, and/or email; through engaging activities including competitions, affirmations, journaling, and games; through live assistance and recommendations from real personal coaches or artificial intelligence (AI) based chatbots; and as a tool to help the plurality of users to practice concepts in real life including energy management tools, focus timers, goal-tracking tools and progress tracking tools.

According to one embodiment herein, the method for personalized motivation management also monitors the progress of the plurality of users, and the recommendations provided are adjusted in real time based on the effectiveness of the previous recommendations. The progress of the plurality of users is constantly monitored and the recommendations are adjusted in real time based on the effectiveness of the previous recommendations. This creates a continuous feedback loop where the method re-evaluates the model and recommends alternative choices for the plurality of users based on their responses to the recommendations and any changes in their behavior and progress toward their goal. Hence, the personalized motivation method acts as a personal automated coach for individuals or an automated assistant for coaches to help guide their employees or customers toward their goals. As people move from one goal to another, the method is able to adapt and continue to provide support and guidance.

According to one embodiment herein, one or more motivation models help to understand the various factors that influence the plurality of users' motivation and to use this understanding to improve performance, productivity, and overall well-being. The one or more motivation models also aim to provide a framework for predicting and adjusting motivation levels. In addition, the one or more motivation models can identify strategies or interventions that can be used to either increase or decrease motivation in different situations. Ultimately, the goal of the one or more motivation models is to better understand and enhance motivation in order to improve the outcomes. Furthermore, the behaviour of the plurality of users is a function of internal motivation factors and external motivation factors. The internal motivation factors comprises psychological needs, aspirations, mindset, advancement, cognitive skills, and energy state. The external motivation factors comprise environment context, rewards, and incentives. Furthermore, the method aims to assist the plurality of users in managing their motivation to achieve their goals, change their behavior, and improve their overall well-being. The method also helps to create a personalized plan which includes the individual's desired goals and a strategy for making progress towards them by effectively managing the energy they invest in achieving those goals. The method takes into consideration the plurality of users' internal motivation factors like psychological needs, mindset, and external motivation factors like environment context, rewards, and recognition.

According to one embodiment herein, the psychological needs include the feeling of belonging, esteem needs, and growth. The feeling of belonging includes acceptance of social groups small or large, need for family, and friends, intimacy, trust, acceptance, and receiving and giving love and affection. The esteem needs includes self-respect and respect from others, need for status, recognition, fame, prestige, and attention. Furthermore, growth includes competence, education, mastery, self-confidence, autonomy, and freedom. In addition, there are several strategies that help to improve the feeling of belonging, self-esteem, and growth needs. For instance, developing and maintaining social connections with others can help to improve feelings of belonging and support. This can include joining clubs or groups, participating in team sports or activities, or volunteering in the community. Furthermore, engaging in activities that promote personal growth and development can help to improve self-esteem and meet growth needs. This can include taking on new challenges, learning new skills, or pursuing hobbies or interests. Similarly, focusing on positive self-talk and reframing negative thoughts can help to improve self-esteem and confidence. Setting and working towards achievable goals can help to improve self-esteem and a sense of accomplishment. Moreover, seeking support from friends, and family, or a professional therapist can be helpful in improving self-esteem and addressing any underlying issues that may be impacting motivation. Also taking care of oneself physically, emotionally, and mentally can help to improve feelings of self-worth and overall well-being.

According to one embodiment herein, the psychological needs are captured using self-assessment questionnaires, and the score gets refined over time based on the insights from the plurality of users interaction. Moreover, the method of measurement of psychological needs includes self-assessment, task completion status, and inferred data from the plurality of user interactions.

According to one embodiment herein, aspiration, an internal motivation factor is responsible for identifying and tracking the various desires, goals, and purposes of the plurality of users. In addition, the method utilizes aspiration to manage and maintain the plurality of users' motivation. The method for measurement of aspiration includes self-assessment, task completion status, and inferred data from the plurality of user interactions. Furthermore, aspiration is a strong desire or ambition to achieve something or to fulfill a particular purpose or goal. It is often used to describe an individual's hopes and dreams for the future, or their aspirations for what they want to achieve in life. Aspiration can be personal, in that it pertains to an individual's own goals and desires, or it can be more collective, in that it relates to the aims and objectives of a group or organization. Aspiration is often driven by a sense of purpose or meaning, and can be a powerful motivator that helps people to set and work towards achieving their goals. In addition, desire is a strong feeling of wanting something or wanting to do something. It is an emotional drive that comes from within an individual and can motivate them to take action. Desire can be positive or negative, depending on the nature of the object or activity that is desired. For example, an individual might have a desire for success, happiness, or adventure, or they might have the desire to avoid failure, pain, or boredom. Moreover, goals, on the other hand, are specific targets or outcomes that individuals set for themselves. They are more concrete and specific than desires and often involve the development of a plan or strategy to achieve the desired outcome. While desire can be a powerful motivator, goals provide direction and focus for that motivation, helping an individual to channel their energy and efforts towards a specific objective. Also, people often confuse their desires with goals, but these are two distinct concepts. The method captures the desires and goals of the plurality of users, recognizes the difference between desire and goal, and helps the plurality of users develop a greater awareness of the role that these concepts play in motivation. The method also provides tools and techniques to help the plurality of users turn their desires into actionable goals. This can include techniques like affirmations to increase the intensity of the individual's desires and help them pursue their goals with greater determination and enthusiasm.

Moreover, the purpose is the reason for which something is done or created or the intention behind an action or decision. Purpose can also refer to the sense of meaning or significance that an individual finds in their life or the goals and values that guide their actions. Purpose can be personal, in that it pertains to an individual's own motivations and aspirations, or it can be more collective, in that it relates to the aims and objectives of a group or organization. In general, purpose is what drives people to take action and helps to give their lives direction and meaning. The strength of a person's motivation is closely tied to the strength of their aspiration. Factors that can impact the strength of a person's aspiration include the clarity of their desire or goal, the frequency with which they think about it, and the emotional intensity that they feel around it. The method can use various ways to influence these factors and help individuals develop stronger aspirations and more powerful motivations.

Furthermore, the strategies to create a purpose, desire, and goal for the plurality of users includes identifying the plurality of users values and goals, appeal to the plurality of users emotions, utilizing positive reinforcement, offering challenges and opportunities for growth, encouraging autonomy, fostering a supportive environment, considering strengths and skills, and reflecting on experiences and challenges.

According to one embodiment herein, the mindset, an internal motivation factor uses belief system, personal values, and personality types to model the baseline of the plurality of users and recommends the plurality of users perform a set of activities and tracks the progress. Based on the progress the method for personalized motivation management refines the recommendations. Furthermore, the method of measurement of mindset includes self-assessment questionnaires, data from other subsystems, and infrared data from the activities, engagements, and journals. In addition, the belief system helps to find the limiting beliefs which limit the plurality of users' ability and limit motivation level to achieve their goals. The method for measurement of the belief system includes self-assessment, task completion status, and inferred data from the plurality of user interactions. Furthermore, personal values help the plurality of users to identify the values and help them live by the values. In addition, the personality helps to provide the innate motivation elements of the plurality of users, and the method recommends the type of coaching based on personality types, data, and tendencies. Furthermore, mindset is a mental framework or set of assumptions that individuals use to interpret and make sense of the world around them. Key factors that can influence an individual's mindset include their belief system, values, and personality type. By understanding and managing these factors, individuals can learn to control their mindset and use it to manage their motivation and behavior.

Moreover, a belief system is a set of beliefs that individuals hold about themselves and the world around them. These beliefs can arise from experiences, influences, and values, and they can shape an individual's behavior and motivation. For example, an individual who has a fixed mindset may believe that their abilities and traits are fixed and cannot be changed, while someone with a growth mindset may believe that they can improve and develop through effort and experience. These beliefs can have a significant impact on the plurality of users' motivation, both at a personal level and in the workplace. For example, a manager who has a fixed mindset may believe that they need to micromanage their employees in order to get good performance, while a manager with a growth mindset may believe that employees are capable of self-direction and will take pride in their achievements if given the opportunity. Similarly, personal values are the fundamental beliefs that guide and motivate an individual's attitudes and actions. The method provides tools and strategies to help individuals identify and strengthen their core values, which can help them manage their motivation and achieve their goals. The method focuses on values in the workplace and family environment for simplicity. Similarly, personality is a set of inherent traits and characteristics that shape the plurality of users behavior and motivations. There are various personality types, each with its own strengths and weaknesses. For example, individuals with a Type-A personality may be highly ambitious and driven, while those with a Type-B personality may be more relaxed and easy-going. Personality traits like conscientiousness, openness, and extraversion have been found to be positively related to intrinsic motivation, while traits like conscientiousness, extraversion, and neuroticism have been found to be positively related to extrinsic achievement motivation. Furthermore, expectations refer to the beliefs and assumptions that an individual holds about the outcomes or consequences of their actions. These expectations can play a significant role in motivation, as they can influence a person's desire to take action and pursue their goals. If an individual has high expectations of the outcomes of their actions, they may be more motivated to put in the necessary effort and work towards achieving their goals. On the other hand, if an individual has low expectations, they may be less motivated to pursue their goals or may not believe that their efforts will lead to success. Additionally, expectations can also be influenced by past experiences and external factors, such as social norms and cultural expectations. For example, if an individual has previously experienced success in a particular area, they may have higher expectations for future success in that area, which can in turn increase their motivation to continue pursuing their goals.

According to one embodiment herein, the energy state is the plurality of users perceived energy level, to achieve their goals and the energy state includes both physical energy and emotional energy of the plurality of users. Cultivating and sustaining heightened levels of energy will help a person to achieve their goals. Key sources of energy includes physical energy and emotional energy. Physical energy renew through sleep and rest, daytime rest, fitness and nutrition. Ways to manage physical energy includes healthy diet, hydration, exercise, sleep, and rest. Furthermore, the emotional energy includes the feelings of enjoyment, caring, satisfaction, and a sense of safety and trust. The emotional energy gets impacted positively and negatively, such as internal conflict due to moral issues, fairness issues, conflict issues, feeling empowered to set boundaries and focus, and sense of meaning and purpose in work.

According to one embodiment herein, the cognitive skill of the plurality of users is assessed based on mindfulness, attention management, emotion regulation, and habit formation to manage motivation, the cognitive skill is tracked to improve the motivation skills of the plurality of users. Certain cognitive skills can significantly impact a person's motivation. These skills allow individuals to observe and potentially see more positive things, as well as regulate their emotions in a way that benefits their motivation. Some examples of cognitive skills that can influence motivation include mindfulness, attention management, emotion regulation, and habit formation. Mindfulness involves being self-aware of one's own mental state, emotions, and goals, as well as being aware of the present moment. This awareness can help to improve motivation by making goals more visible and attainable. For example, if you are mindful of your own energy levels, you can plan tasks that fit your current energy level and utilize your motivation to achieve them. When you are successful in achieving your goals, it can give you more confidence and increase your motivation. Additionally, mindfulness can help with awareness of distractions and the ability to regulate emotions in the face of distractions, which can improve motivation by allowing you to stay focused and on track. Overall, mindfulness can be a helpful tool in improving motivation and goal achievement. Furthermore, attention management is the ability to manage attention to the desired activities. Strategies include identifying distractions and eliminating them, creating a distraction-free environment, setting clear goals and priorities, using time-blocking and taking breaks. In addition, emotion regulation is the ability to regulate emotions, involves having knowledge and skills to manage and cope with a range of emotions. This capability can be especially important in maintaining motivation, as negative emotions can drain motivation and make it difficult to achieve goals. The techniques for regulating emotions, includes labeling and reframing, helps to manage motivation and stay on track towards your goals. For example, using stress management and emotional regulation techniques can help to reframe stressful situations in a more peaceful and manageable way, which can improve motivation. Overall, the ability to regulate emotions can be a valuable skill in maintaining motivation and achieve goals. Furthermore, habit formation is automatic actions that we perform without conscious thought. Forming positive habits can be a powerful way to improve motivation. For example, if you have established a habit of exercising in the morning or meditating regularly, these automatic actions can give you a boost in motivation. These habits can help to create a positive mental environment that supports and nourishes your motivation. Overall, establishing positive habits can be a helpful strategy in fostering and maintaining motivation.

According to one embodiment herein, the advancement is the plurality of users feelings about the progress made by them towards their goal and value. Personal growth or recognition at work or school is a recognition of their progress. When people feel they are progressing, they gain higher motivation. When the progress is made with fun, then this creates a much higher level of motivation. When the necessary job is part of the desire, this creates long-term motivation. Motivation can be sustained by gamifying (making it fun) for every task. Furthermore, the method offers gamification options to the plurality of users as advancement, an internal motivation factor. Hence, achievement can have a big impact on a person's motivation. When we achieve our goals, it can give us a sense of accomplishment and pride, which can in turn increase our motivation to continue pursuing our goals and aspirations. On the other hand, if we are unable to achieve our goals, it can lead to disappointment and a lack of motivation. Furthermore, achievement can have a positive impact on confidence. When we achieve our goals, which can boost our self-confidence. This increased confidence can then give us the motivation and determination to take on new challenges and continue pursuing our goals. In addition, the method keeps track of the progress and achievement made by the person regardless of whether the progress or achievement is big or small, and uses this periodically to boost motivation when the system notices the emotional energy level is low. The method also recommends people to formulate the goals for the desire and break down the goals into smaller chunks and have clear progress tracking on small parts. When the person sees they complete each small component, their confidence goes up in the expectation of the achievement which improves motivation.

According to one embodiment herein, the environment context recommends strategies to change the context and measure the impact using the change in energy level. The context includes physical, social and informational. The physical context includes weather, food the user eats, and the place the user lives, the social context includes friends, family, and co-worker, and the informational context includes culture, policies, and process. Hence, environment context is one of the key motivational elements that influence the motivation of a person. This factor can be easily controlled. The physical environment context includes weather or climate, physically built environment—community resources like parks, nutrition environment, and neighborhood. The social environment context includes social support, norms, belief and attitudes. Furthermore, the information environment includes policies, and processes, especially in the work environment.

According to one embodiment herein, the method also tracks the plurality of users rewards and incentives to accelerate the motivation level of the plurality of users. Further, the plurality of users uses the rewards and incentives to develop their own personal development system, which aligns with the goals to accelerate motivation. Rewards improve motivation in several ways, such as rewards provide a sense of accomplishment, rewards can increase intrinsic motivation, rewards can increase effort and rewards can increase engagement. Hence, it is important to consider the individual's goals and motivation style when using rewards to improve motivation, as different rewards may be more or less effective for different people. It is also important to use rewards appropriately, as overuse or misuse of rewards can lead to a decrease in motivation. The rewards can be of several types, such as verbal praise, physical rewards, social recognition, monetary awards, opportunities for advancement, and time off or flexible working hours or location.

According to one embodiment herein, the method for managing the one or more motivation models is provided. The method comprises creating a motivation baseline for the plurality of users through questionnaires, skill assessments, activities, and response to activities. The method further includes creating an improvement plan based on the motivation baseline. Furthermore, the method includes gamifying the improvement plan for each of the plurality of users and engaging with the plurality of users through a game. For an individual user, the game is a single-player game and for a company, the game is a multi-player game with a plurality of employees or a single-player game, for an individual employee. For instance, a company is prompted to create games involving leaderboards, to enable a plurality of employees to compete in a fun way. Furthermore, for a company, an individual employee is also enabled to create a game for themselves, so that an employee tries to finish a feature coding and complete it within preset hours of time such as in a period of 2 hours. Thus the employee is enabled to set a time and complete the task by oneself. The company is provided with a single-player-game as well as multi-player games. The method further includes measuring continuously and refining strategies for uplifting motivation and providing insights to the plurality of users on the current state and progress. In addition, the method includes learning from the similar plurality of users and incorporating learning back to the general population. Also, the method includes managing motivation levels across various groups and taking strategic action within a company, and managing individually using a single application for each of the plurality of users. Furthermore, the method includes personalizing various levels including individual level, corporate level, or based on a coach's input. The method further includes understanding the concepts based on knowledge content. The knowledge content is a content repository comprising images, tiny tips cards, letters, emails, chat messages, videos, and presentations. In addition, the method includes using a combination of coach and an individual user as a multi-user usage.

According to one embodiment herein, the self-assessment questions includes input provided by the plurality of user as a set of questions. Furthermore, the user activity data is collected by using an application, website, or third-party application providing insight into the plurality of users' motivation and related factors. Furthermore, the motivation score is generated based on the plurality of motivation factors and sub-factors. The motivation score is the product of the individual factor score and the weightage of the factor. The weightage of the factor is in the range of 0 to 10 and the motivation score is categorized as low (<25% score), medium (>25% and <75%), and high (>75%).

According to one embodiment herein, engaging the plurality of users includes through a just-in-time recommendation or a tip through a card as the plurality of users enter data, displaying motivating images or quotes to uplift the plurality of users, offering options to the plurality of users to personalize the applications and the content the plurality of users viewing, using in-app alerts to share pieces of content or insights, sharing content with others by the plurality of users, sending small tips to the plurality of users through messaging applications including WhatsApp, slack, and Microsoft® Teams, using an interactive bot to provide insights to the plurality of users and collecting input and feedback.

According to one embodiment herein, learning from the similar plurality of users includes best practices based on the user personas and patterns. The best practices are given more weightage and recommendations across the similar plurality of users. Furthermore, the method also helps in personalizing various levels by customizing and setting preferences for the tip cards, images, and messages entered by the plurality of users to improve their mood.

According to one embodiment herein, a computer-implemented system for personalized motivation management is provided. The system comprises an attribute extraction module configured to extract a plurality of motivation attributes from a plurality of users through external applications, websites, and external devices the plurality of users interact with, and additionally collecting external data through an application programming interface. The plurality of motivation attributes includes the user's current motivation level, behavior, skill level, and desired goals. Furthermore, the system comprises a motivation modelling module configured to generate one or more motivation models for each of the plurality of users based on motivation modeling criteria derived from a plurality of motivation factors. The plurality of motivation factors comprises internal motivation factors and external motivation factors. The internal motivation factors include psychological needs, aspirations, mindset, advancement, cognitive skills, and energy state and the external motivation factors include environment context, rewards and incentives. In addition, the system comprises a motivation score generating module configured to generate a motivation score for each of the plurality of users by measuring the plurality of user's motivation and scoring the plurality of motivation factors and associated plurality of motivation sub-factors. The plurality of user's motivation is measured through self-assessment questions, system calculation, and user activity data. Furthermore, the system comprises a motivation management subsystem module configured to dynamically generate one or more targeted, control elements specific to a target user and render the one or more targeted, control elements on a user device of the target user through a plurality of delivery channels, among the plurality of users using the one or more motivation models generated by the motivation modelling module, the motivation score generated by the motivation score generating module, and one or more content libraries. Furthermore, the user device comprises a smartwatch, smartphone, computer, and/or laptop; and wherein the plurality of delivery channels include smartwatches, smartphones, applications in smartwatches and smartphones, bots including Microsoft® Teams, and web interfaces. The one or more targeted, control elements include the plurality of motivation factors, influencing the plurality of users' behavior and help in achieving the motivation goal.

According to one embodiment herein, the attribute extraction module is also configured to extract the plurality of motivation attributes from the plurality of users through a questionnaire. The external applications include chatbot, mobile applications, web interface, and/or web portal and the external devices include smart watches, smartphones, computers, and/or laptops.

According to one embodiment herein, the motivation modelling module configured to generate one or more motivation models is a digital twin of the plurality of users, which represents the plurality of users' motivation level. The system further assists the plurality of users in recognizing the user's motivation level at that moment and maintains the motivation necessary to accomplish the plurality of users' goals. In addition, the motivation modelling module configured to generate one or more motivation models serve as a model of the desired future state by providing visibility and insights into the current state, and by offering recommendations through a recommendation module for the future state. The recommendation module in conjunction with the motivation modelling module configured to generate recommendations for the future state comprises range of knowledge content and skill-building activities for the plurality of users to achieving their goals. Furthermore, the recommendations for the future state are delivered to the plurality of users as a micro-content delivered directly through the applications or through channels including WhatsApp, slack, and/or email; through engaging activities including competitions, affirmations, journaling, and games; through live assistance and recommendations from real personal coaches or artificial intelligence (AI) based chatbots; and as a tool to help the plurality of users to practice concepts in real life including energy management tools, focus timers, goal-tracking tools and progress tracking tools.

According to one embodiment herein, the system further monitors the progress of the plurality of users, and the recommendations provided by the recommendation module in conjunction with the motivation modelling module is adjusted in real-time based on the effectiveness of the previous recommendations. Furthermore, the motivation modelling module configured to generate the one or more motivation models help to understand the various factors that influence the plurality of users' motivation and to use this understanding to improve performance, productivity, and overall well-being. The one or more motivation models also aim to provide a framework for predicting and adjusting motivation levels. In addition, the one or more motivation models also identifies strategies or interventions used to either increase or decrease motivation in different situations and the one or more motivation models further helps to understand and enhance motivation in order to improve the outcomes.

According to one embodiment herein, psychological needs include the feeling of belonging, esteem needs, and growth. The feeling of belonging includes acceptance of social groups small or large, need for family, and friends, intimacy, trust, acceptance, and receiving and giving love and affection. Further, the esteem needs include self-respect and respect from others, need for status, recognition, fame, prestige, and attention. In addition, the growth includes competence, education, mastery, self-confidence, autonomy, and freedom; The psychological needs are captured using self-assessment questionnaires and the score gets refined over time based on the insights from the plurality of user's interaction. Further, the method of measurement of psychological needs includes self-assessment, task completion status, and inferred data from the plurality of user interactions.

According to one embodiment herein, aspiration, an internal motivation factor is responsible for identifying and tracking the various desires, goals, and purposes of the plurality of users. The system utilizes aspiration to manage and maintain the plurality of users motivation and the method of measurement of aspiration include self-assessment, task completion status, inferred data from the plurality of user interactions.

According to one embodiment herein, the mindset, an internal motivation factor uses belief system, personal values, and personality types to model the baseline of the plurality of users and recommends the plurality of users, to perform a set of activities and tracks the progress. Based on the progress, the system refines the recommendations and the method of measurement of mindset includes self-assessment questionnaires, data from other subsystems, and infrared data from the activities, engagements, and journals. Furthermore, the belief system helps to find the limiting beliefs which limit the plurality of users' ability and limit motivation level to achieve their goals. The method of measurement of the belief system includes self-assessment, task completion status, inferred data from the plurality of user interactions. Further, the personal values helps the plurality of users to identify the values and help them live by the values and the personality helps to provide the innate motivation elements of the plurality of users. The system further recommends the type of coaching based on personality types, data, and tendencies.

According to one embodiment herein, the cognitive skill of the plurality of users is assessed based on mindfulness, attention management, emotion regulation, and habit formation to manage motivation. The cognitive skill is tracked to improve the motivation skills of the plurality of users. Furthermore, the system also offers gamification options to the plurality of users as advancement, an internal motivation factor. In addition, the environment context recommends strategies to change the context and measure the impact using the change in energy level. The context includes physical, social and informational. The physical context includes weather, food the user eats, and the place the user lives, the social context includes friends, family, and co-workers and the informational context includes culture, policies, and process.

According to one embodiment herein, the system is also configured to track the plurality of users rewards and incentives to accelerate the motivation level of the plurality of users. The plurality of users uses the rewards and incentives to develop their own personal development system, that aligns with the goals to accelerate motivation.

According to one embodiment herein, the method for managing the motivation modelling module configured to generate one or more motivation models is provided. The method comprises creating a motivation baseline for the plurality of users through a questionnaire, skill assessment, activities, and response to activities. The method further includes creating an improvement plan based on the motivation baseline. Furthermore, the method includes gamifying the improvement plan for each of the plurality of users and engaging with the plurality of users through a game. For an individual user, the game is a single-player game. For a company, the game is a multi-player game with a plurality of employees or a single-player game, when an individual employee is involved. For instance, a company can create games involving leaderboards, to enable different people to compete in a fun way. Furthermore, the company is also provided with an option to enable an individual employee to create a game for themselves in a company, so that the individual employee is provided with an option to finish/complete a feature coding within 2 hours of time. Hence the employee is enabled to set a time and complete the task themselves. Thus the company can have single-player-game as well as multi-player games. The method further includes measuring continuously and refining strategies for uplifting motivation and providing insights to the plurality of users on the current state and progress. In addition, the method includes learning from the similar plurality of users and incorporating learning back to the general population. Also, the method includes managing motivation levels across various groups and taking strategic action within a company, and managing individually using a single application for each of the plurality of users. Furthermore, the method includes personalizing various levels including individual level, corporate level, or based on a coach's input. The method further includes understanding the concepts based on knowledge content. The knowledge content is a content repository comprising images, tiny tips cards, letters, emails, chat messages, videos, and presentations. In addition, the method includes using a combination of coach and an individual user as a multi-user usage.

According to one embodiment herein, the self-assessment questions includes input provided by the plurality of user as a set of questions and the user activity data is collected through using an application, website, or third-party applications providing insight into the plurality of users' motivation and related factors.

According to one embodiment herein, the motivation score generating module configured to generate motivation score is generated based on the plurality of motivation factors and sub-factors. The motivation score is the product of individual factor score and weightage of the factor and the weightage of the factor is in the range of 0 to 10. Furthermore, the motivation score is categorized as low (<25% score), medium (>25% and <75%), and high (>75%).

According to one embodiment herein, the improvement plan includes providing tiny tips, comprising small pieces of educational content to improve the lowest score of the plurality of users. Furthermore, engaging the plurality of users includes a just-in-time recommendation or a tip through a card as the plurality of users enters data, displaying motivating images or quotes to uplift the plurality of users, offering options to the plurality of users to personalize the applications and the content the plurality of users viewing, using in-app alerts to share pieces of content or insights, sharing content with others by the plurality of users, sending small tips to the plurality of users through messaging applications including WhatsApp, slack, and Microsoft® Teams, using an interactive bot to provide insights to the plurality of users and collecting input and feedback. Furthermore, the learning from the similar plurality of users includes best practices based on the user personas and patterns. The best practices are given more weightage and recommendations across the similar plurality of users. In addition, the personalizing various levels includes customizing and setting preferences for the tip cards, images and messages entered by the plurality of users to improve their mood.

The foregoing summary is illustrative only and is not intended to be in any way limiting. In addition to the illustrative aspects, embodiments, and features described above, further aspects, embodiments, and features will become apparent by reference to the drawings and the following detailed description.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flowchart depicting a method for personalized motivation management, according to one embodiment herein.

FIG. 2 illustrates an exemplary high-level decomposition of a computer-implemented system for personalized motivation management, according to an embodiment herein.

FIG. 3 illustrates a functional block diagram of a personalized motivation management system, according to one embodiment herein.

FIG. 4 illustrates a graph depicting time vs motivation using the system for personalized motivation management, according to one embodiment herein.

FIG. 5A-5J illustrates the list of attributes of the plurality of motivation attributes, according to an embodiment herein.

FIG. 6A-6I exemplarily illustrates screenshots of user interfaces of personalized motivation management platform, according to an embodiment herein.

FIG. 7 illustrates a sample motivation score card generated for a user, according to an embodiment herein.

Although the specific features of the present invention are shown in some drawings and not in others. This is done for convenience only as each feature may be combined with any or all of the other features in accordance with the present invention.

### DETAILED DESCRIPTION OF THE EMBODIMENTS HEREIN

In the following detailed description, reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical, and other changes may be made without departing from the scope of the embodiments. The following detailed description is therefore not to be taken in a limiting sense.

The foregoing of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

The various embodiments herein provide a method and system for personalized motivation management. The method and system for personalized motivation management help to manage motivation in a plurality of users to improve the plurality of users, productivity, and well-being. Furthermore, the system for personalized motivation management also helps the plurality of users to manage their motivation and achieve their goals, thereby increasing motivation throughout the day. In addition, the system for personalized serves as the basis for the continuous collection of various data elements, including several categories of information, with numerous subcategories and factors. The system is configured to gather data using a questionnaire, applications, websites, and devices the plurality of users engage with to provide effective motivation models.

According to one embodiment herein, a method for personalized motivation management is provided. The method comprises extracting a plurality of motivation attributes from a plurality of users, through external applications, websites, and external devices the plurality of users interact with, and additionally collecting external data through an application programming interface. The plurality of motivation attributes includes the user's current motivation level, behavior, skill level, and desired goals. The method further comprises dynamically generating one or more motivation models for each of the plurality of users based on motivation modeling criteria derived from a plurality of motivation factors. The plurality of motivation factors comprises internal motivation factors and external motivation factors. The internal motivation factors include psychological needs, aspirations, mindset, advancement, cognitive skills, and energy state and the external motivation factors include environment context, rewards, and incentives. Furthermore, the method comprises generating a motivation score for each of the plurality of users by measuring the plurality of users' motivation and scoring the plurality of motivation factors and associated plurality of motivation sub-factors. The plurality of user's motivation is measured through self-assessment questions, system calculation, and user activity data. In addition, the method includes dynamically generating one or more targeted, control elements specific to a target user, among the plurality of users using one or more motivation models, the motivation score, and one or more content libraries. The one or more targeted, control elements include the plurality of motivation factors influencing the plurality of users' behavior and help in achieving the motivation goal. Finally, the method includes rendering the one or more targeted, control elements on a user device of the target user through a plurality of delivery channels. Furthermore, the user device comprises a smartwatch, smartphone, computer, and/or laptop. The plurality of delivery channels include smartwatches, smartphones, applications in smartwatches and smartphones, bots including Microsoft® Teams, and web interfaces.

According to one embodiment herein, the plurality of motivation attributes from the plurality of users is also extracted by the method through a questionnaire. Moreover, the external applications include chatbots, mobile applications, web interfaces, and/or web portals and the external devices include smart watches, smartphones, computers, and/or laptops. The chatbots give an interactive experience for the plurality of users working with the platform. The plurality of users can type in questions and the chatbot provides guidance and relevant charts/details related to motivation. The mobile applications are easy-to-use applications for the plurality of users to interact with the system to enter the data. Furthermore, the web interfaces are an alternative to mobile applications, easy for blog reading, and help to review various reports.

According to one embodiment herein, one or more motivation models is a digital twin of the plurality of users, which represents the plurality of users' motivation level. Furthermore, the method for personalized motivation management also assists the plurality of users in recognizing the user's motivation level at that moment and maintaining the motivation necessary to accomplish the plurality of users' goals. The plurality of users includes an individual user, a company, and or a lifestyle coach.

According to one embodiment herein, one or more motivation models generated by the method for personalized motivation management serve as a model of the desired future state by providing visibility and insights into the current state, and by offering recommendations for the future state. The recommendations for the future state comprise a range of knowledge content and skill-building activities for the plurality of users to achieve their goals. Furthermore, the recommendations for the future state are delivered to the plurality of users as a micro-content delivered directly through the applications or through channels including WhatsApp, slack, and/or email; through engaging activities including competitions, affirmations, journaling, and games; through live assistance and recommendations from real personal coaches or artificial intelligence (AI) based chatbots; and as a tool to help the plurality of users to practice concepts in real life including energy management tools, focus timers, goal-tracking tools and progress tracking tools.

According to one embodiment herein, the method for personalized motivation management also monitors the progress of the plurality of users, and the recommendations provided are adjusted in real time based on the effectiveness of the previous recommendations. The progress of the plurality of users is constantly monitored and the recommendations are adjusted in real time based on the effectiveness of the previous recommendations. This creates a continuous feedback loop where the method re-evaluates the model and recommends alternative choices for the plurality of users based on their responses to the recommendations and any changes in their behavior and progress toward their goal. Hence, the personalized motivation method acts as a personal automated coach for individuals or an automated assistant for coaches to help guide their employees or customers toward their goals. As people move from one goal to another, the method is able to adapt and continue to provide support and guidance.

According to one embodiment herein, one or more motivation models help to understand the various factors that influence the plurality of users' motivation and to use this understanding to improve performance, productivity, and overall well-being. The one or more motivation models also aim to provide a framework for predicting and adjusting motivation levels. In addition, the one or more motivation models can identify strategies or interventions that can be used to either increase or decrease motivation in different situations. Ultimately, the goal of the one or more motivation models is to better understand and enhance motivation in order to improve the outcomes. Furthermore, the behaviour of the plurality of users is a function of internal motivation factors and external motivation factors. The internal motivation factors comprises psychological needs, aspirations, mindset, advancement, cognitive skills, and energy state. The external motivation factors comprise environment context, rewards, and incentives. Furthermore, the method aims to assist the plurality of users in managing their motivation to achieve their goals, change their behavior, and improve their overall well-being. The method also helps to create a personalized plan which includes the individual's desired goals and a strategy for making progress towards them by effectively managing the energy they invest in achieving those goals. The method takes into consideration the plurality of users' internal motivation factors like psychological needs, mindset, and external motivation factors like environment context, rewards, and recognition.

According to one embodiment herein, the psychological needs include the feeling of belonging, esteem needs, and growth. The feeling of belonging includes acceptance of social groups small or large, need for family, and friends, intimacy, trust, acceptance, and receiving and giving love and affection. The esteem needs includes self-respect and respect from others, need for status, recognition, fame, prestige, and attention. Furthermore, growth includes competence, education, mastery, self-confidence, autonomy, and freedom. In addition, there are several strategies that help to improve the feeling of belonging, self-esteem, and growth needs. For instance, developing and maintaining social connections with others can help to improve feelings of belonging and support. This can include joining clubs or groups, participating in team sports or activities, or volunteering in the community. Furthermore, engaging in activities that promote personal growth and development can help to improve self-esteem and meet growth needs. This can include taking on new challenges, learning new skills, or pursuing hobbies or interests. Similarly, focusing on positive self-talk and reframing negative thoughts can help to improve self-esteem and confidence. Setting and working towards achievable goals can help to improve self-esteem and a sense of accomplishment. Moreover, seeking support from friends, and family, or a professional therapist can be helpful in improving self-esteem and addressing any underlying issues that may be impacting motivation. Also taking care of oneself physically, emotionally, and mentally can help to improve feelings of self-worth and overall well-being.

According to one embodiment herein, the psychological needs are captured using self-assessment questionnaires, and the score gets refined over time based on the insights from the plurality of users interaction. Moreover, the method of measurement of psychological needs includes self-assessment, task completion status, and inferred data from the plurality of user interactions.

According to one embodiment herein, aspiration, an internal motivation factor is responsible for identifying and tracking the various desires, goals, and purposes of the plurality of users. In addition, the method utilizes aspiration to manage and maintain the plurality of users' motivation. The method for measurement of aspiration includes self-assessment, task completion status, and inferred data from the plurality of user interactions. Furthermore, aspiration is a strong desire or ambition to achieve something or to fulfill a particular purpose or goal. It is often used to describe an individual's hopes and dreams for the future, or their aspirations for what they want to achieve in life. Aspiration can be personal, in that it pertains to an individual's own goals and desires, or it can be more collective, in that it relates to the aims and objectives of a group or organization. Aspiration is often driven by a sense of purpose or meaning, and can be a powerful motivator that helps people to set and work towards achieving their goals. In addition, desire is a strong feeling of wanting something or wanting to do something. It is an emotional drive that comes from within an individual and can motivate them to take action. Desire can be positive or negative, depending on the nature of the object or activity that is desired. For example, an individual might have a desire for success, happiness, or adventure, or they might have the desire to avoid failure, pain, or boredom. Moreover, goals, on the other hand, are specific targets or outcomes that individuals set for themselves. They are more concrete and specific than desires and often involve the development of a plan or strategy to achieve the desired outcome. While desire can be a powerful motivator, goals provide direction and focus for that motivation, helping an individual to channel their energy and efforts towards a specific objective. Also, people often confuse their desires with goals, but these are two distinct concepts. The method captures the desires and goals of the plurality of users, recognizes the difference between desire and goal, and helps the plurality of users develop a greater awareness of the role that these concepts play in motivation. The method also provides tools and techniques to help the plurality of users turn their desires into actionable goals. This can include techniques like affirmations to increase the intensity of the individual's desires and help them pursue their goals with greater determination and enthusiasm.

Moreover, the purpose is the reason for which something is done or created or the intention behind an action or decision. Purpose can also refer to the sense of meaning or significance that an individual finds in their life or the goals and values that guide their actions. Purpose can be personal, in that it pertains to an individual's own motivations and aspirations, or it can be more collective, in that it relates to the aims and objectives of a group or organization. In general, purpose is what drives people to take action and helps to give their lives direction and meaning. The strength of a person's motivation is closely tied to the strength of their aspiration. Factors that can impact the strength of a person's aspiration include the clarity of their desire or goal, the frequency with which they think about it, and the emotional intensity that they feel around it. The method can use various ways to influence these factors and help individuals develop stronger aspirations and more powerful motivations.

Furthermore, the strategies to create a purpose, desire, and goal for the plurality of users includes identifying the plurality of users values and goals, appeal to the plurality of users emotions, utilizing positive reinforcement, offering challenges and opportunities for growth, encouraging autonomy, fostering a supportive environment, considering strengths and skills, and reflecting on experiences and challenges.

According to one embodiment herein, the mindset, an internal motivation factor uses belief system, personal values, and personality types to model the baseline of the plurality of users and recommends the plurality of users perform a set of activities and tracks the progress. Based on the progress the method for personalized motivation management refines the recommendations. Furthermore, the method of measurement of mindset includes self-assessment questionnaires, data from other subsystems, and infrared data from the activities, engagements, and journals. In addition, the belief system helps to find the limiting beliefs which limit the plurality of users' ability and limit motivation level to achieve their goals. The method for measurement of the belief system includes self-assessment, task completion status, and inferred data from the plurality of user interactions. Furthermore, personal values help the plurality of users to identify the values and help them live by the values. In addition, the personality helps to provide the innate motivation elements of the plurality of users, and the method recommends the type of coaching based on personality types, data, and tendencies. Furthermore, mindset is a mental framework or set of assumptions that individuals use to interpret and make sense of the world around them. Key factors that can influence an individual's mindset include their belief system, values, and personality type. By understanding and managing these factors, individuals can learn to control their mindset and use it to manage their motivation and behavior.

Moreover, a belief system is a set of beliefs that individuals hold about themselves and the world around them. These beliefs can arise from experiences, influences, and values, and they can shape an individual's behavior and motivation. For example, an individual who has a fixed mindset may believe that their abilities and traits are fixed and cannot be changed, while someone with a growth mindset may believe that they can improve and develop through effort and experience. These beliefs can have a significant impact on the plurality of users' motivation, both at a personal level and in the workplace. For example, a manager who has a fixed mindset may believe that they need to micromanage their employees in order to get good performance, while a manager with a growth mindset may believe that employees are capable of self-direction and will take pride in their achievements if given the opportunity. Similarly, personal values are the fundamental beliefs that guide and motivate an individual's attitudes and actions. The method provides tools and strategies to help individuals identify and strengthen their core values, which can help them manage their motivation and achieve their goals. The method focuses on values in the workplace and family environment for simplicity. Similarly, personality is a set of inherent traits and characteristics that shape the plurality of users behavior and motivations. There are various personality types, each with its own strengths and weaknesses. For example, individuals with a Type-A personality may be highly ambitious and driven, while those with a Type-B personality may be more relaxed and easy-going. Personality traits like conscientiousness, openness, and extraversion have been found to be positively related to intrinsic motivation, while traits like conscientiousness, extraversion, and neuroticism have been found to be positively related to extrinsic achievement motivation. Furthermore, expectations refer to the beliefs and assumptions that an individual holds about the outcomes or consequences of their actions. These expectations can play a significant role in motivation, as they can influence a person's desire to take action and pursue their goals. If an individual has high expectations of the outcomes of their actions, they may be more motivated to put in the necessary effort and work towards achieving their goals. On the other hand, if an individual has low expectations, they may be less motivated to pursue their goals or may not believe that their efforts will lead to success. Additionally, expectations can also be influenced by past experiences and external factors, such as social norms and cultural expectations. For example, if an individual has previously experienced success in a particular area, they may have higher expectations for future success in that area, which can in turn increase their motivation to continue pursuing their goals.

According to one embodiment herein, the energy state is the plurality of users perceived energy level, to achieve their goals and the energy state includes both physical energy and emotional energy of the plurality of users. Cultivating and sustaining heightened levels of energy will help a person to achieve their goals. Key sources of energy includes physical energy and emotional energy. Physical energy renew through sleep and rest, daytime rest, fitness and nutrition. Ways to manage physical energy includes healthy diet, hydration, exercise, sleep, and rest. Furthermore, the emotional energy includes the feelings of enjoyment, caring, satisfaction, and a sense of safety and trust. The emotional energy gets impacted positively and negatively, such as internal conflict due to moral issues, fairness issues, conflict issues, feeling empowered to set boundaries and focus, and sense of meaning and purpose in work.

According to one embodiment herein, the cognitive skill of the plurality of users is assessed based on mindfulness, attention management, emotion regulation, and habit formation to manage motivation, the cognitive skill is tracked to improve the motivation skills of the plurality of users. Certain cognitive skills can significantly impact a person's motivation. These skills allow individuals to observe and potentially see more positive things, as well as regulate their emotions in a way that benefits their motivation. Some examples of cognitive skills that can influence motivation include mindfulness, attention management, emotion regulation, and habit formation. Mindfulness involves being self-aware of one's own mental state, emotions, and goals, as well as being aware of the present moment. This awareness can help to improve motivation by making goals more visible and attainable. For example, if you are mindful of your own energy levels, you can plan tasks that fit your current energy level and utilize your motivation to achieve them. When you are successful in achieving your goals, it can give you more confidence and increase your motivation. Additionally, mindfulness can help with awareness of distractions and the ability to regulate emotions in the face of distractions, which can improve motivation by allowing you to stay focused and on track. Overall, mindfulness can be a helpful tool in improving motivation and goal achievement. Furthermore, attention management is the ability to manage attention to the desired activities. Strategies include identifying distractions and eliminating them, creating a distraction-free environment, setting clear goals and priorities, using time-blocking and taking breaks. In addition, emotion regulation is the ability to regulate emotions, involves having knowledge and skills to manage and cope with a range of emotions. This capability can be especially important in maintaining motivation, as negative emotions can drain motivation and make it difficult to achieve goals. The techniques for regulating emotions, includes labeling and reframing, helps to manage motivation and stay on track towards your goals. For example, using stress management and emotional regulation techniques can help to reframe stressful situations in a more peaceful and manageable way, which can improve motivation. Overall, the ability to regulate emotions can be a valuable skill in maintaining motivation and achieve goals. Furthermore, habit formation is automatic actions that we perform without conscious thought. Forming positive habits can be a powerful way to improve motivation. For example, if you have established a habit of exercising in the morning or meditating regularly, these automatic actions can give you a boost in motivation. These habits can help to create a positive mental environment that supports and nourishes your motivation. Overall, establishing positive habits can be a helpful strategy in fostering and maintaining motivation.

According to one embodiment herein, the advancement is the plurality of users feelings about the progress, they are making towards their goal and value. Personal growth or recognition at work or school is a recognition of their progress. When people feel they are progressing, they gain higher motivation. When the progress is made with fun, then this creates a much higher level of motivation. When the necessary job is part of the desire, this creates long-term motivation. Motivation can be sustained by gamifying (making it fun) for every task. Furthermore, the method offers gamification options to the plurality of users as advancement, an internal motivation factor. Hence, achievement can have a big impact on a person's motivation. When we achieve our goals, it can give us a sense of accomplishment and pride, which can in turn increase our motivation to continue pursuing our goals and aspirations. On the other hand, if we are unable to achieve our goals, it can lead to disappointment and a lack of motivation. Furthermore, achievement can have a positive impact on confidence. When we achieve our goals, which can boost our self-confidence. This increased confidence can then give us the motivation and determination to take on new challenges and continue pursuing our goals. In addition, the method keeps track of the progress and achievement the person is making—big or small and uses this periodically to boost motivation when the system notices the emotional energy level is low. The method also recommends people to formulate the goals for the desire and break down the goals into smaller chunks and have clear progress tracking on small parts. When the person sees they complete each small component, their confidence goes up in the expectation of the achievement which improves motivation.

According to one embodiment herein, the environment context recommends strategies to change the context and measure the impact using the change in energy level. The context includes physical, social and informational. The physical context includes weather, food the user eats, and the place the user lives, the social context includes friends, family, and co-worker, and the informational context includes culture, policies, and process. Hence, environment context is one of the key motivational elements that influence the motivation of a person. This factor can be easily controlled. The physical environment context includes weather or climate, physically built environment—community resources like parks, nutrition environment, and neighborhood. The social environment context includes social support, norms, belief and attitudes. Furthermore, the information environment includes policies, and processes, especially in the work environment.

According to one embodiment herein, the method also tracks the plurality of users rewards and incentives to accelerate the motivation level of the plurality of users. Further, the plurality of users uses the rewards and incentives to develop their own personal development system, which aligns with the goals to accelerate motivation. Rewards improve motivation in several ways, such as rewards provide a sense of accomplishment, rewards can increase intrinsic motivation, rewards can increase effort and rewards can increase engagement. Hence, it is important to consider the individual's goals and motivation style when using rewards to improve motivation, as different rewards may be more or less effective for different people. It is also important to use rewards appropriately, as overuse or misuse of rewards can lead to a decrease in motivation. The rewards can be of several types, such as verbal praise, physical rewards, social recognition, monetary awards, opportunities for advancement, and time off or flexible working hours or location.

According to one embodiment herein, the method for managing the one or more motivation models is provided. The method comprises creating a motivation baseline for the plurality of users through questionnaires, skill assessments, activities and response to activities. The method further includes creating an improvement plan based on the motivation baseline. Furthermore, the method includes gamifying the improvement plan for each of the plurality of users and engaging with the plurality of users through a game. For an individual user the game is a single-player game and for a company, the game is a multi-player game with a plurality of employees or a single-player game, when an individual employee is involved. For instance, a company can create games involving leaderboards, to enable different people to compete in a fun way. Furthermore, the company is also provided with an option to enable an individual employee to create a game for themselves in a company, so that the individual employee is provided with an option to finish/complete a feature coding within 2 hours of time. Hence the employee is enabled to set a time and complete the task themselves. Thus the company can have single-player-game as well as multi-player games. The method further includes measuring continuously and refining strategies for uplifting motivation and providing insights to the plurality of users on the current state and progress. In addition, the method includes learning from the similar plurality of users and incorporating learning back to the general population. Also, the method includes managing motivation levels across various groups and taking strategic action within a company, and managing individually using a single application for each of the plurality of users. Furthermore, the method includes personalizing various levels including individual level, corporate level, or based on a coach's input. The method further includes understanding the concepts based on knowledge content. The knowledge content is a content repository comprising images, tiny tips cards, letters, emails, chat messages, videos, and presentations. In addition, the method includes using a combination of coach and an individual user as a multi-user usage.

According to one embodiment herein, the self-assessment questions includes input provided by the plurality of user as a set of questions. Furthermore, the user activity data is collected by using an application, website, or third-party application providing insight into the plurality of users' motivation and related factors. Furthermore, the motivation score is generated based on the plurality of motivation factors and sub-factors. The motivation score is the product of the individual factor score and the weightage of the factor. The weightage of the factor is in the range of 0 to 10 and the motivation score is categorized as low (<25% score), medium (>25% and <75%), and high (>75%).

In addition, there are several factors and attributes that can impact a person's motivation, and these are measured to create a baseline motivation score for an individual. Some examples of these factors and attributes are provided in Table 1.

TABLE 1

| Motivation Factors | Scoring Attributes |
|---|---|
| Psychological Needs | Family_socre |
| | Friendship_score |
| | Intimacy_score |
| | Acceptance_score |
| | Trusted_relationship_score |
| | Love_score |
| | Feeling_status_score |
| | Fame_score |
| | Competance_score |
| | Self_cofidence_score |
| | Independance_score |
| Aspiration -Desire, | Have_desire |
| Goals & Purpose | Have_desire_to_goal_created |
| | Have_goal_dev_skill |
| | Have_goal_focus_skill |
| | Desire_list (id, desc, type) |
| | Goal_description |
| | Goal_clarity_desc |
| | Goal_Accountability_desc |
| | Goal_values_def |
| | Goal_timelines |
| | Goal_status |
| | Goal_priority |
| | Goal_breakdown_List |
| | Goal_Gaming_list |
| | Goal_stall_reason_list |
| | Goal_type |
| Mindset | Belief Systems Attributes: |
| | Belief_list |
| | Limiting_belief_list |
| | Fixed_mindset_belief_list |
| | Positive_affirmation_list |
| | Belief_Adpative_score |
| | Belief_awareness_score |
| | Belief_accuracy_assement_score |
| | Positive_Affirmation_used_desc |
| | Personal Values Attributes |
| | Core_value_list |
| | Core_value_action_score_list |
| | Value_assement_score |
| | Value_related_journal_notes |
| | Value_goal_tracking |
| | Value_mindset_affirmation_tracking |
| | Value_assement_score |
| | Value_related_journal_notes |
| | Value_goal_tracking |
| | Value_mindset_affirmation_tracking |
| | Personality |
| | Myer-Briggs_Type |
| | Big_Five_type |
| | MBTI_type |
| | DISC_type |
| | Value_related_journal_notes |
| | Value_goal_tracking |
| | Value_mindset_affirmation_tracking |
| Energy State | Emotional energy (e-battery) level Daily/hourly |
| | measurement |
| | How many times to enter the battery data how |
| | often they see tips cards |
| | Have they read the content provided as the |
| | knowledge Base |
| | Actions performed based on the content |
| | Visibility to their trend and insights from the |
| | Trend- Actions they are performing based on that. |
| Cognitive Skill | Self-assessment score of Current skill for |
| | Mindfulness, Attention management score, |
| | Emotion regulation & Habits formation score. |
| | Activity score for each of the skills |
| | Usage of various tools for each skills |
| Advancement- | Progress chart for each tasks |
| Progress, | Progress person is making for each of the goals/ |
| achievement, and fun | Tasks |
| | Gamification strategy for each task |
| | Measure their ability to create a small task. |
| | Ability to create fun elements for each task. |
| Environment | Self-Assessed Environment score for Physical, |
| Context | Social and Information environment. |
| | Response to the Strategy recommendations |
| Rewards & | Self-assessment of rewards and Incentives |
| Incentives | Response to strategy recommendation |

According to one embodiment herein, the improvement plan includes providing tiny tips, comprising small pieces of educational content to improve the lowest score of the plurality of users. The method analyzes scores for various factors and helps individuals improve their lowest scores by providing tiny tips, which are small pieces of educational content. The plurality of users constantly interacts with the method, either by inputting data or voluntarily reviewing the content. During these interactions, the method delivers tiny tips. For example, if an individual has a low score in the belief system and uses a tool such as the energy tracker, the method may deliver Tiny Tips cards every time they log their energy.

According to one embodiment herein, engaging the plurality of users includes through a just-in-time recommendation or a tip through a card as the plurality of users enter data, displaying motivating images or quotes to uplift the plurality of users, offering options to the plurality of users to personalize the applications and the content the plurality of users viewing, using in-app alerts to share pieces of content or insights, sharing content with others by the plurality of users, sending small tips to the plurality of users through messaging applications including WhatsApp, slack, and Microsoft® Teams, using an interactive bot to provide insights to the plurality of users and collecting input and feedback. Furthermore, the insights or reports provided by the method is as given in the Table 2.

TABLE 2

| | | Insights trends- |
|---|---|---|
| | Insights- data | text insight |
| Factors | and reports | and charts |
| Psychological | Which needs are strong | Progress tracking of |
| needs | based on questionnaire | the Need level. Ex: |
| | input. | not satisfy to satisfy |
| | Goals which has | state |
| | connection with the | |
| | desire needs | |
| Desire | Strength of the desire or | Strength changes over |
| | goal | time of the desire or |
| | Skill level of managing | goal |
| | the desire | |
| | Connection of micro | |
| | goals to the big desire | |
| Mindset | List of self-limiting | Progress on |
| | beliefs | eliminating limiting |
| | Skill level of managing | beliefs |
| | those | |
| | Connection between | |
| | personality and limiting | |
| | beliefs | |
| | Expectation spectrum - | |
| | where they stand on | |
| | expectation compare | |
| | with others | |
| Energy | Avg Energy for a day/ | Change in Energy over |
| | week/Month | a period of time: 7 |
| | Key reason for low | week trend, monthly, |
| | energy | yearly trend. |
| | Key reason for high | Comparison to last |
| | energy | week |
| | | Comparison to similar |
| | | days |
| Skills | Score of each skills | Progress over time to |
| | require for motivation | master the skill |
| | management | |
| | Key strengths and | |
| | weakness | |
| Advancement | Progress tracking of each | Achievement trend - |
| | goal. | correlation with |
| | Gamification aspect of | energy level |
| | each activity | Insights on types of |
| | Ability to break down | achievements to focus |
| | tasks | on what energy time |
| | Measure skill - how | period |
| | many times data is | |
| | entered | |
| | Review and refine - how | |
| | many metrics viewed | |
| | Gamification skill - how | |
| | many gamification | |
| | metrics created | |
| Environment | List of environment | Trend over success of |
| | factors influencing their | environment setting |
| | behavior and motivation | |
| Rewards | Insights of which | Goal and rewards |
| | rewards work for them - | correlation |
| | reward preferences | |
| Over all | Overall well-being of the | Trend over period |
| | person - Motivation | |
| | score (calculated in | |
| | weekly basis) | |

According to one embodiment herein, learning from the similar plurality of users includes best practices based on the user personas and patterns. The best practices are given more weightage and recommendations across the similar plurality of users. Furthermore, the method also helps in personalizing various levels by customizing and setting preferences for the tip cards, images, and messages entered by the plurality of users to improve their mood.

According to one embodiment herein, a computer-implemented system for personalized motivation management is provided. The system comprises an attribute extraction module configured to extract a plurality of motivation attributes from a plurality of users through external applications, websites, and external devices the plurality of users interact with, and additionally collecting external data through an application programming interface. The plurality of motivation attributes includes the user's current motivation level, behavior, skill level, and desired goals. Furthermore, the system comprises a motivation modelling module configured to generate one or more motivation models for each of the plurality of users based on motivation modeling criteria derived from a plurality of motivation factors. The plurality of motivation factors comprises internal motivation factors and external motivation factors. The internal motivation factors include psychological needs, aspirations, mindset, advancement, cognitive skills, and energy state and the external motivation factors include environment context, rewards and incentives. In addition, the system comprises a motivation score generating module configured to generate a motivation score for each of the plurality of users by measuring the plurality of user's motivation and scoring the plurality of motivation factors and associated plurality of motivation sub-factors. The plurality of user's motivation is measured through self-assessment questions, system calculation, and user activity data. Furthermore, the system comprises a motivation management subsystem module configured to dynamically generate one or more targeted, control elements specific to a target user and render the one or more targeted, control elements on a user device of the target user through a plurality of delivery channels, among the plurality of users using the one or more motivation models generated by the motivation modelling module, the motivation score generated by the motivation score generating module, and one or more content libraries. Furthermore, the user device comprises a smartwatch, smartphone, computer, and/or laptop; and wherein the plurality of delivery channels include smartwatches, smartphones, applications in smartwatches and smartphones, bots including Microsoft® Teams, and web interfaces. The one or more targeted, control elements include the plurality of motivation factors, influencing the plurality of users' behavior and help in achieving the motivation goal.

According to one embodiment herein, the attribute extraction module is also configured to extract the plurality of motivation attributes from the plurality of users through a questionnaire. The external applications include chatbot, mobile applications, web interface, and/or web portal and the external devices include smart watches, smartphones, computers, and/or laptops.

According to one embodiment herein, the motivation modelling module configured to generate one or more motivation models is a digital twin of the plurality of users, which represents the plurality of users' motivation level. The system further assists the plurality of users in recognizing the user's motivation level at that moment and maintains the motivation necessary to accomplish the plurality of users' goals. In addition, the motivation modelling module configured to generate one or more motivation models serve as a model of the desired future state by providing visibility and insights into the current state, and by offering recommendations through a recommendation module for the future state. The recommendation module in conjunction with the motivation modelling module configured to generate recommendations for the future state comprises range of knowledge content and skill-building activities for the plurality of users to achieving their goals. Furthermore, the recommendations for the future state are delivered to the plurality of users as a micro-content delivered directly through the applications or through channels including WhatsApp, slack, and/or email; through engaging activities including competitions, affirmations, journaling, and games; through live assistance and recommendations from real personal coaches or artificial intelligence (AI) based chatbots; and as a tool to help the plurality of users to practice concepts in real life including energy management tools, focus timers, goal-tracking tools and progress tracking tools.

According to one embodiment herein, the system further monitors the progress of the plurality of users, and the recommendations provided by the recommendation module in conjunction with the motivation modelling module is adjusted in real-time based on the effectiveness of the previous recommendations. Furthermore, the motivation modelling module configured to generate the one or more motivation models help to understand the various factors that influence the plurality of users' motivation and to use this understanding to improve performance, productivity, and overall well-being. The one or more motivation models also aim to provide a framework for predicting and adjusting motivation levels. In addition, the one or more motivation models also identifies strategies or interventions used to either increase or decrease motivation in different situations and the one or more motivation models further helps to understand and enhance motivation in order to improve the outcomes.

According to one embodiment herein, psychological needs include the feeling of belonging, esteem needs, and growth. The feeling of belonging includes acceptance of social groups small or large, need for family, and friends, intimacy, trust, acceptance, and receiving and giving love and affection. Further, the esteem needs include self-respect and respect from others, need for status, recognition, fame, prestige, and attention. In addition, the growth includes competence, education, mastery, self-confidence, autonomy, and freedom; The psychological needs are captured using self-assessment questionnaires and the score gets refined over time based on the insights from the plurality of user's interaction. Further, the method of measurement of psychological needs includes self-assessment, task completion status, and inferred data from the plurality of user interactions.

According to one embodiment herein, aspiration, an internal motivation factor is responsible for identifying and tracking the various desires, goals, and purposes of the plurality of users. The system utilizes aspiration to manage and maintain the plurality of users motivation and the method of measurement of aspiration include self-assessment, task completion status, inferred data from the plurality of user interactions.

According to one embodiment herein, the mindset, an internal motivation factor uses belief system, personal values, and personality types to model the baseline of the plurality of users and recommends the plurality of users, to perform a set of activities and tracks the progress. Based on the progress, the system refines the recommendations and the method of measurement of mindset includes self-assessment questionnaires, data from other subsystems, and infrared data from the activities, engagements, and journals. Furthermore, the belief system helps to find the limiting beliefs which limit the plurality of users' ability and limit motivation level to achieve their goals. The method of measurement of the belief system includes self-assessment, task completion status, inferred data from the plurality of user interactions. Further, the personal values helps the plurality of users to identify the values and help them live by the values and the personality helps to provide the innate motivation elements of the plurality of users. The system further recommends the type of coaching based on personality types, data, and tendencies.

According to one embodiment herein, the cognitive skill of the plurality of users is assessed based on mindfulness, attention management, emotion regulation, and habit formation to manage motivation. The cognitive skill is tracked to improve the motivation skills of the plurality of users. Furthermore, the system also offers gamification options to the plurality of users as advancement, an internal motivation factor. In addition, the environment context recommends strategies to change the context and measure the impact using the change in energy level. The context includes physical, social and informational. The physical context includes weather, food the user eats, and the place the user lives, the social context includes friends, family, and co-workers and the informational context includes culture, policies, and process.

According to one embodiment herein, the system is also configured to track the plurality of users rewards and incentives to accelerate the motivation level of the plurality of users. The plurality of users uses the rewards and incentives to develop their own personal development system, that aligns with the goals to accelerate motivation.

According to one embodiment herein, the method for managing the motivation modelling module configured to generate one or more motivation models is provided. The method comprises creating a motivation baseline for the plurality of users through a questionnaire, skill assessment, activities, and response to activities. The method further includes creating an improvement plan based on the motivation baseline. Furthermore, the method includes gamifying the improvement plan for each of the plurality of users and engaging with the plurality of users through a game. For an individual user, the game is a single-player game and for a company, the game is a multi-player game with a plurality of employees or a single-player game, when an individual employee is involved. For instance, a company is provided with an option to create games involving leaderboards, to enable different people to compete in a fun way. Furthermore, the company is also provided with an option to enable an individual employee to create a game for themselves in a company, so that the individual employee is provided with an option to develop and/complete a feature coding within 2 hours of time. Hence the employee is enabled to set a time and complete the task themselves. Thus the company can have single-player-game as well as multi-player games. The method further includes measuring continuously and refining strategies for uplifting motivation and providing insights to the plurality of users on the current state and progress. In addition, the method includes learning from the similar plurality of users and incorporating learning back to the general population. Also, the method includes managing motivation levels across various groups and taking strategic action within a company, and managing individually using a single application for each of the plurality of users. Furthermore, the method includes personalizing various levels including individual level, corporate level, or based on a coach's input. The method further includes understanding the concepts based on knowledge content. The knowledge content is a content repository comprising images, tiny tips cards, letters, emails, chat messages, videos, and presentations. In addition, the method includes using a combination of coach and an individual user as a multi-user usage.

According to one embodiment herein, the self-assessment questions includes input provided by the plurality of user as a set of questions and the user activity data is collected through using an application, website, or third-party applications providing insight into the plurality of users' motivation and related factors.

According to one embodiment herein, the motivation score generating module configured to generate motivation score is generated based on the plurality of motivation factors and sub-factors. The motivation score is the product of individual factor score and weightage of the factor and the weightage of the factor is in the range of 0 to 10. Furthermore, the motivation score is categorized as low (<25% score), medium (>25% and <75%), and high (>75%).

According to one embodiment herein, the improvement plan includes providing tiny tips, comprising small pieces of educational content to improve the lowest score of the plurality of users. Furthermore, engaging the plurality of users includes a just-in-time recommendation or a tip through a card as the plurality of users enters data, displaying motivating images or quotes to uplift the plurality of users, offering options to the plurality of users to personalize the applications and the content the plurality of users viewing, using in-app alerts to share pieces of content or insights, sharing content with others by the plurality of users, sending small tips to the plurality of users through messaging applications including WhatsApp, slack, and Microsoft® Teams, using an interactive bot to provide insights to the plurality of users and collecting input and feedback. Furthermore, the learning from the similar plurality of users includes best practices based on the user personas and patterns. The best practices are given more weightage and recommendations across the similar plurality of users. In addition, the personalizing various levels includes customizing and setting preferences for the tip cards, images and messages entered by the plurality of users to improve their mood.

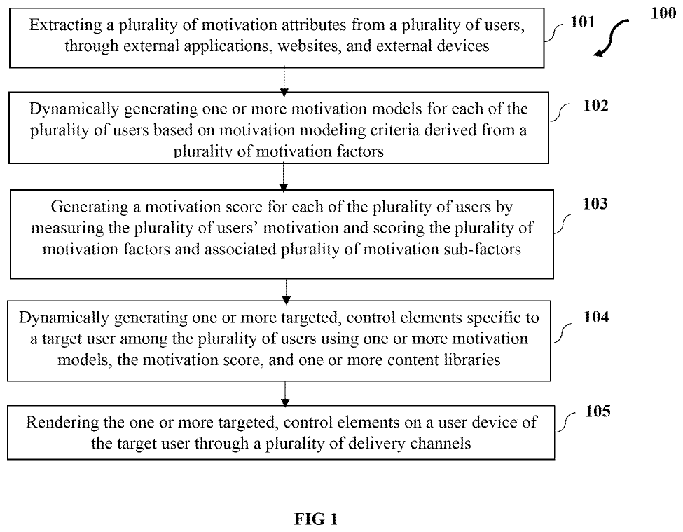

FIG. 1 illustrates a flowchart depicting a method for personalized motivation management, according to one embodiment herein. The method 100 comprises extracting a plurality of motivation attributes from a plurality of users, through external applications, websites, and external devices the plurality of users interact with, and additionally collecting external data through an application programming interface at step 101. The plurality of motivation attributes includes the user's current motivation level, behavior, skill level, and desired goals. The method 100 further comprises dynamically generating one or more motivation models for each of the plurality of users based on motivation modeling criteria derived from a plurality of motivation factors at step 102. The plurality of motivation factors comprises internal motivation factors and external motivation factors. The internal motivation factors include psychological needs, aspirations, mindset, advancement, cognitive skills, and energy state and the external motivation factors include environment context, rewards, and incentives. Furthermore, the method 100 comprises generating a motivation score for each of the plurality of users by measuring the plurality of users' motivation and scoring the plurality of motivation factors and associated plurality of motivation sub-factors at step 103. The plurality of user's motivation is measured through self-assessment questions, system calculation, and user activity data. In addition, the method 100 includes dynamically generating one or more targeted, control elements specific to a target user, among the plurality of users using one or more motivation models, the motivation score, and one or more content libraries at step 104. The one or more targeted, control elements include the plurality of motivation factors influencing the plurality of users' behavior and help in achieving the motivation goal. Finally, the method 100 includes rendering the one or more targeted, control elements on a user device of the target user through a plurality of delivery channels at step 105. Furthermore, the user device comprises a smartwatch, smartphone, computer, and/or laptop. The plurality of delivery channels include smartwatches, smartphones, applications in smartwatches and smartphones, bots including Microsoft® Teams, and web interfaces.

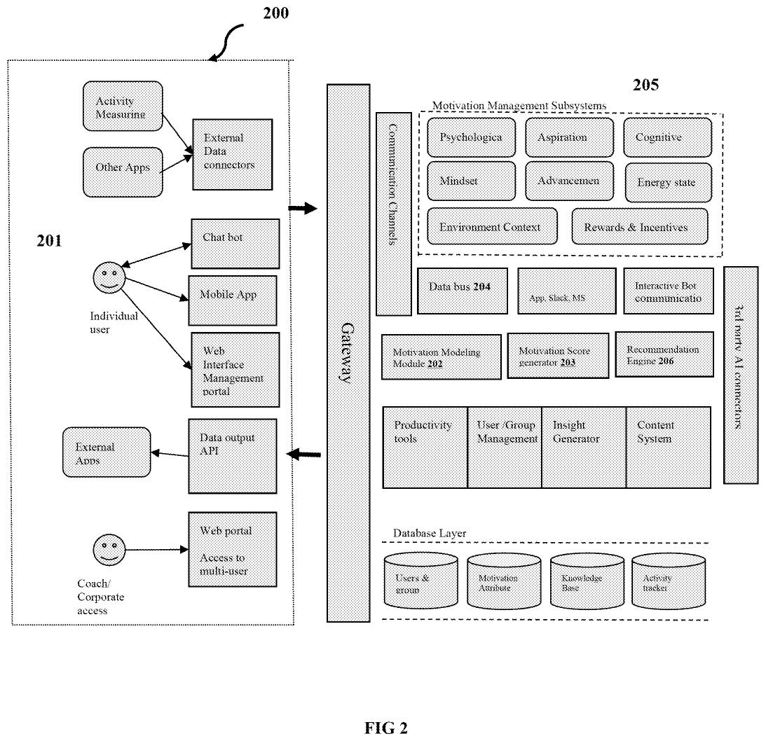

FIG. 2 illustrates an exemplary high-level decomposition of a computer-implemented system for personalized motivation management, according to an embodiment herein. The system 200 comprises an attribute extraction module 201 configured to extract a plurality of motivation attributes from a plurality of users through external applications, websites, and external devices the plurality of users interact with, and additionally collecting external data through an application programming interface. The plurality of motivation attributes includes the user's current motivation level, behavior, skill level, and desired goals. Furthermore, the system 200 comprises a motivation modelling module 202 configured to generate one or more motivation models for each of the plurality of users based on motivation modeling criteria derived from a plurality of motivation factors. The plurality of motivation factors comprises internal motivation factors and external motivation factors. The internal motivation factors include psychological needs, aspirations, mindset, advancement, cognitive skills, and energy state and the external motivation factors include environment context, rewards, and incentives. In addition, the system 200 comprises a motivation score generating module 203 configured to generate a motivation score for each of the plurality of users by measuring the plurality of user's motivation and scoring the plurality of motivation factors and associated plurality of motivation sub-factors. The plurality of user's motivation is measured through self-assessment questions, system calculation, and user activity data. Furthermore, the system 200 comprises a data bus 204 to collect external data and transfer it to various subsystem logics. Furthermore, the system 200 comprises a motivation management subsystem module 205 configured to dynamically generate one or more targeted, control elements specific to a target user and render the one or more targeted, control elements on a user device of the target user through a plurality of delivery channels, among the plurality of users using the one or more motivation models generated by the motivation modelling module 203, the motivation score generated by the motivation score generating module 204, and one or more content libraries. Furthermore, the user device comprises a smartwatch, smartphone, computer, and/or laptop; and wherein the plurality of delivery channels include smartwatches, smartphones, applications in smartwatches and smartphones, bots including Microsoft® Teams, and web interfaces. The one or more targeted, control elements include the plurality of motivation factors, influencing the plurality of users' behavior and help in achieving the motivation goal. In addition, the system 200 comprises a recommendation module 206, which works in conjunction with the motivation modelling module 203 configured to generate recommendations for the future state comprises range of knowledge content and skill-building activities for the plurality of users to achieving their goals.

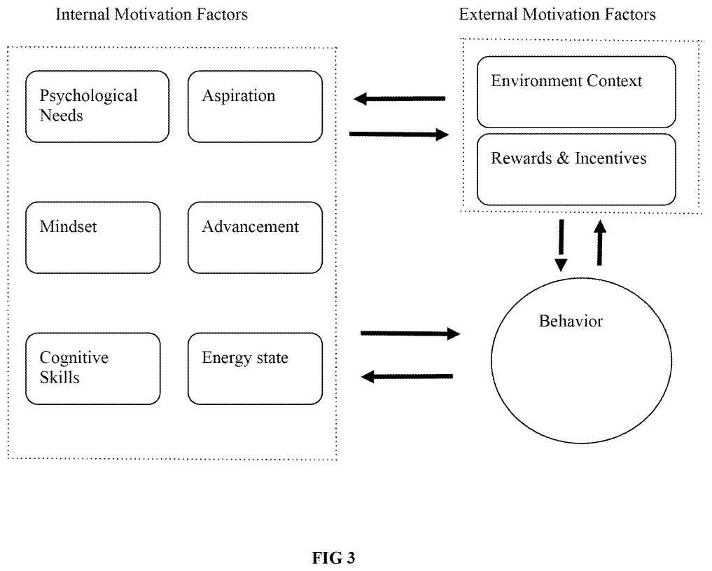

FIG. 3 illustrates a functional block diagram of personalized motivation management system, according to one embodiment herein. FIG. 3 illustrates the personalized motivation management system comprising a plurality of motivation factors influencing the behaviour of a user. The plurality of motivation factors further comprises internal motivation factors and external motivation factors. The internal motivation factors include psychological needs, aspirations, mindset, advancement, cognitive skills, and energy state and the external motivation factors include environment context, rewards, and incentives.

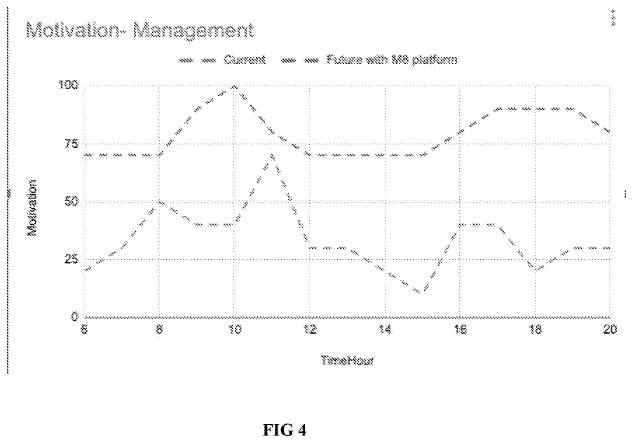

FIG. 4 illustrates a graph depicting time vs motivation using the system for personalized motivation management, according to one embodiment herein. FIG. 4 depicts the graph showing changes in motivation with time using the embodiments herein. In FIG. 4, the blue line shows the typical motivation score of a user without employing the embodiments herein and the red line shows the motivation score of a user employing the embodiments herein, and overall improvement in the motivation level.

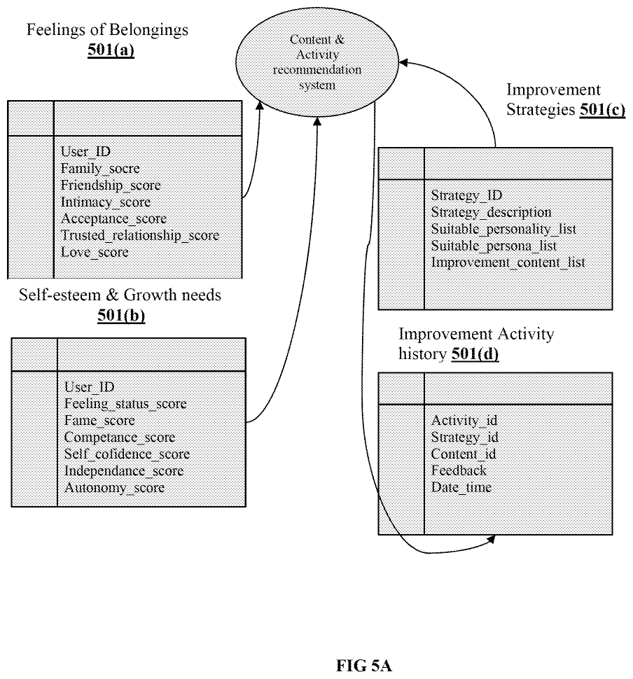

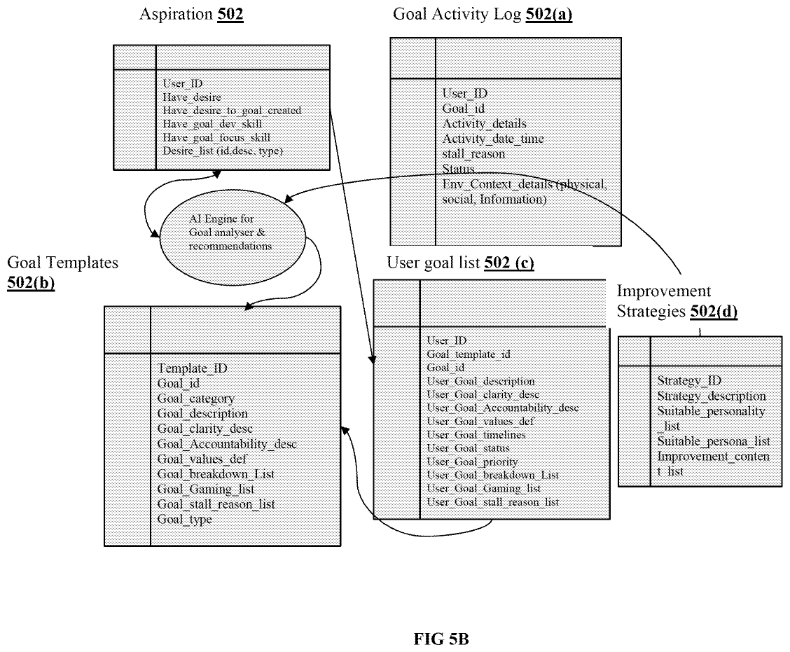

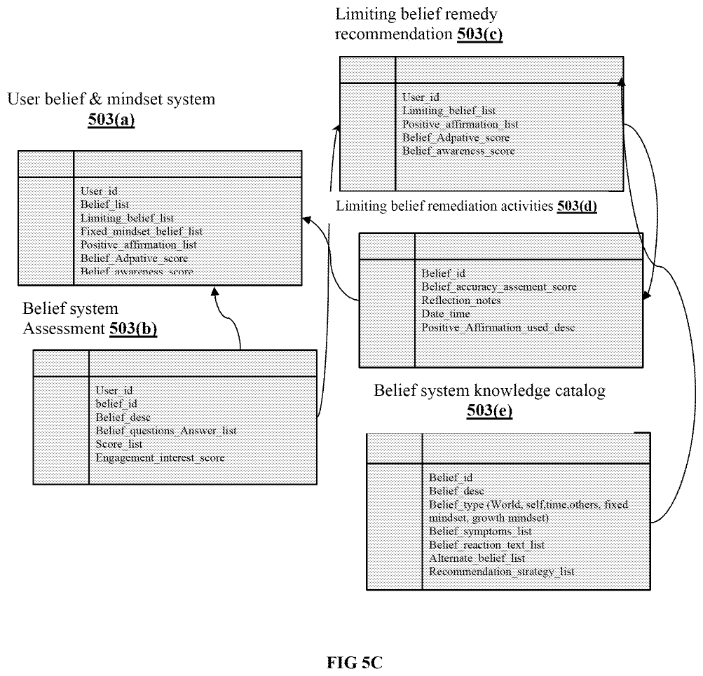

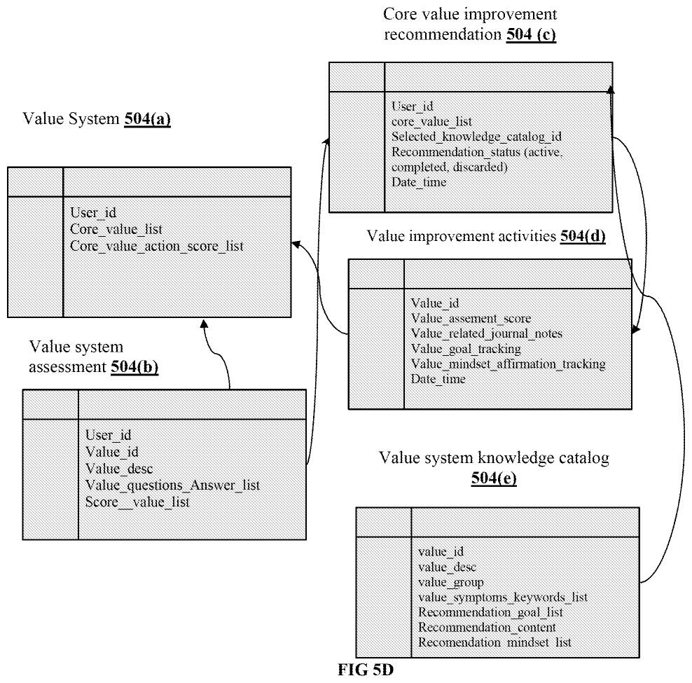

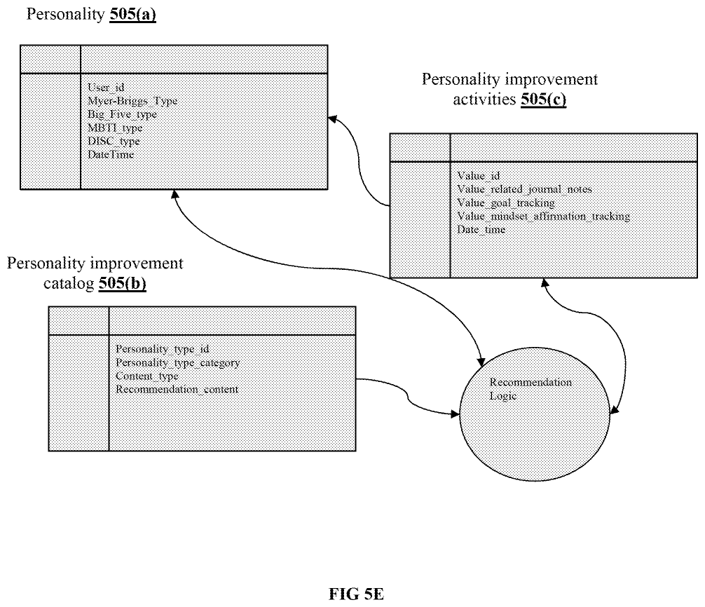

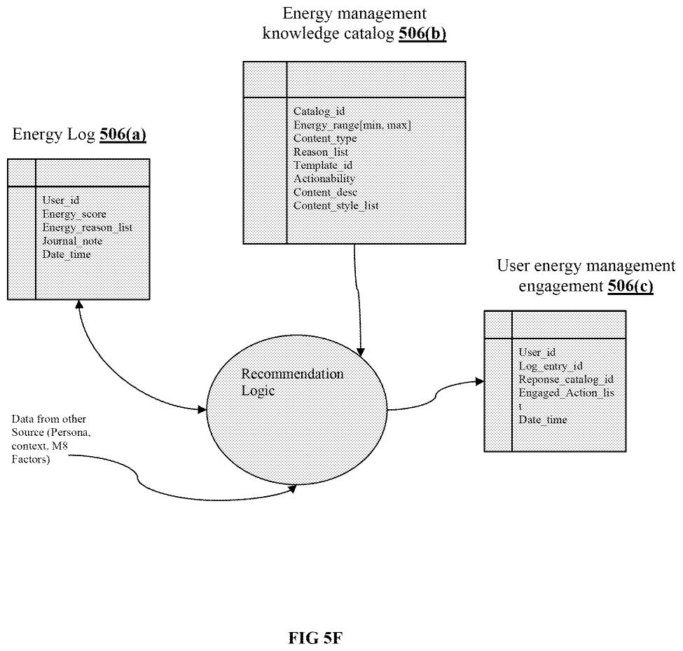

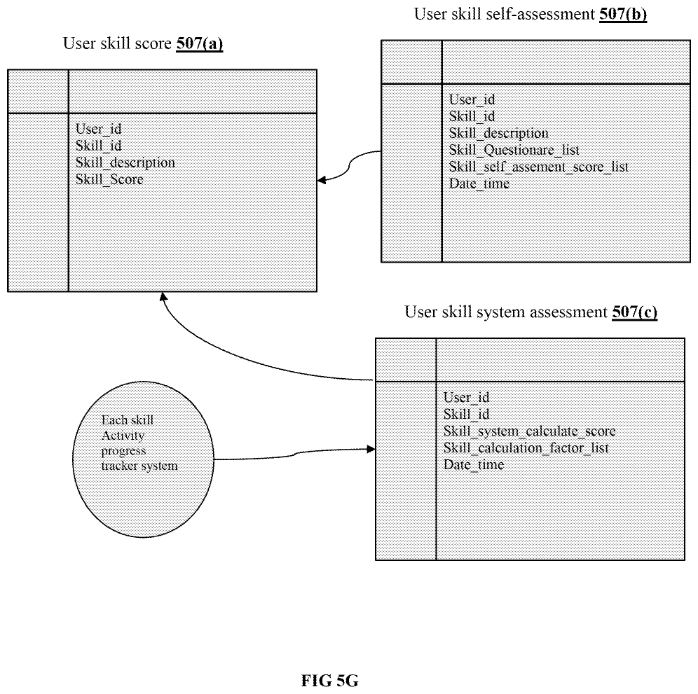

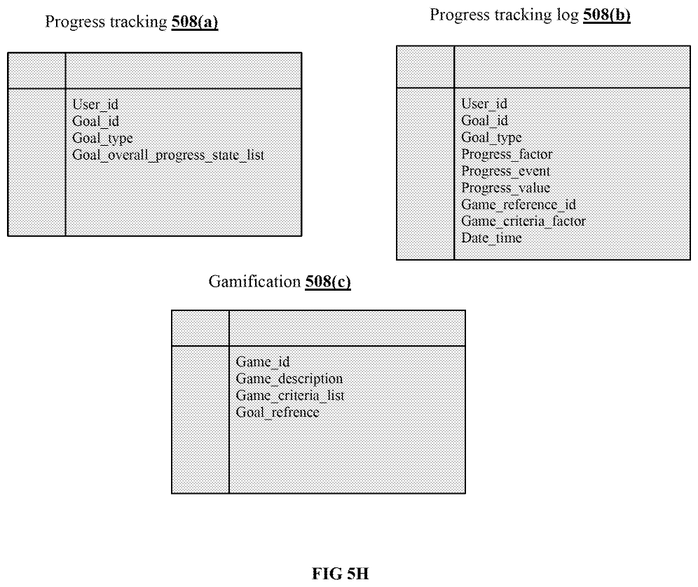

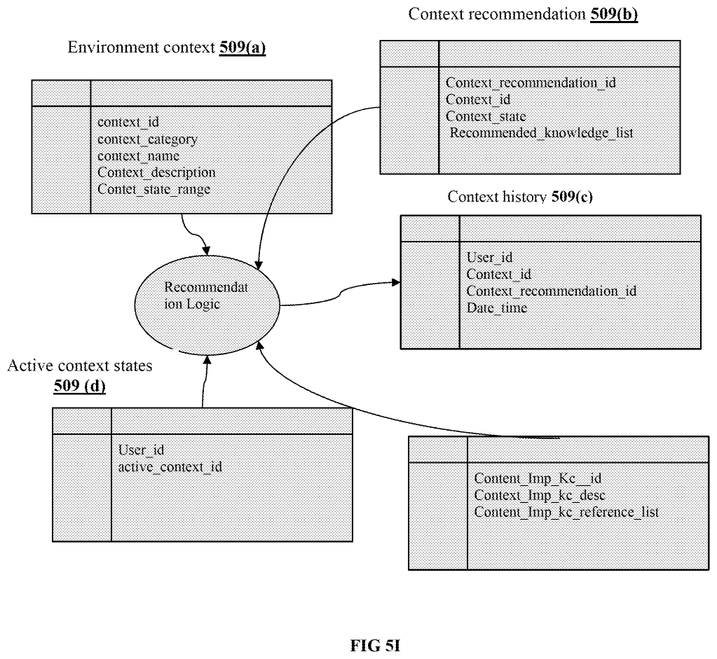

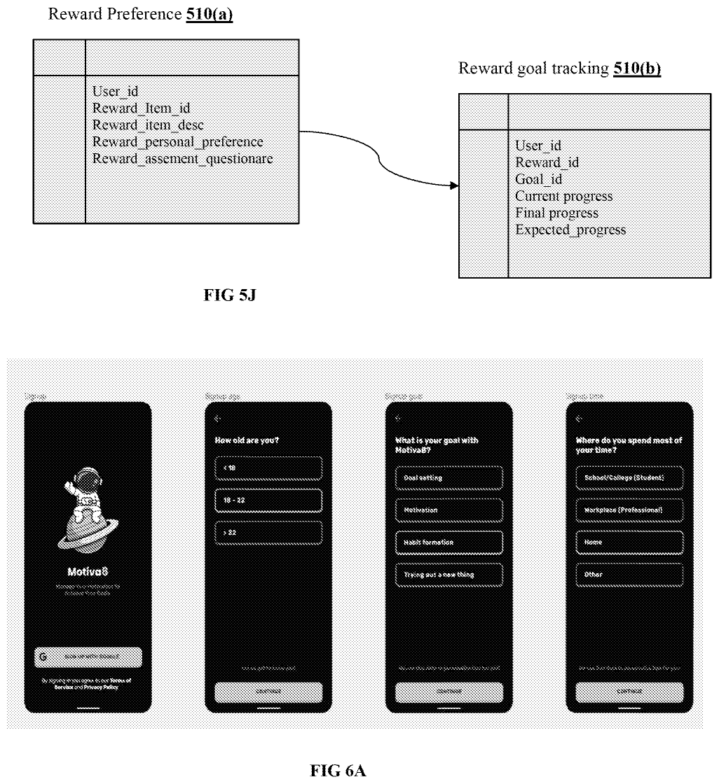

FIG. 5A-5J illustrates the list of attributes of the plurality of motivation attributes, according to an embodiment herein. FIG. 5A illustrates the list of attributes of the psychological needs, according to an embodiment herein. The psychological needs include the feeling of belonging, esteem needs, and growth. The feeling of belonging includes acceptance of social groups small or large, need for family, and friends, intimacy, trust, acceptance, and receiving and giving love and affection. The esteem needs includes self-respect and respect from others, need for status, recognition, fame, prestige, and attention. Furthermore, growth includes competence, education, mastery, self-confidence, autonomy, and freedom. In addition, there are several strategies that help to improve the feeling of belonging, self-esteem, and growth needs. For instance, developing and maintaining social connections with others can help to improve feelings of belonging and support. This can include joining clubs or groups, participating in team sports or activities, or volunteering in the community. Furthermore, engaging in activities that promote personal growth and development can help to improve self-esteem and meet growth needs. This can include taking on new challenges, learning new skills, or pursuing hobbies or interests. Similarly, focusing on positive self-talk and reframing negative thoughts can help to improve self-esteem and confidence. Setting and working towards achievable goals can help to improve self-esteem and a sense of accomplishment. Moreover, seeking support from friends, and family, or a professional therapist can be helpful in improving self-esteem and addressing any underlying issues that may be impacting motivation. Also taking care of oneself physically, emotionally, and mentally can help to improve feelings of self-worth and overall well-being.

FIG. 5B illustrates the list of attributes of the aspiration, according to an embodiment herein. aspiration, an internal motivation factor is responsible for identifying and tracking the various desires, goals, and purposes of the plurality of users. In addition, the method utilizes aspiration to manage and maintain the plurality of users' motivation. The method for measurement of aspiration includes self-assessment, task completion status, and inferred data from the plurality of user interactions. Furthermore, aspiration is a strong desire or ambition to achieve something or to fulfill a particular purpose or goal. It is often used to describe an individual's hopes and dreams for the future, or their aspirations for what they want to achieve in life. Aspiration can be personal, in that it pertains to an individual's own goals and desires, or it can be more collective, in that it relates to the aims and objectives of a group or organization. Aspiration is often driven by a sense of purpose or meaning, and can be a powerful motivator that helps people to set and work towards achieving their goals. In addition, desire is a strong feeling of wanting something or wanting to do something. It is an emotional drive that comes from within an individual and can motivate them to take action. Desire can be positive or negative, depending on the nature of the object or activity that is desired. For example, an individual might have a desire for success, happiness, or adventure, or they might have the desire to avoid failure, pain, or boredom. Moreover, goals, on the other hand, are specific targets or outcomes that individuals set for themselves. They are more concrete and specific than desires and often involve the development of a plan or strategy to achieve the desired outcome. While desire can be a powerful motivator, goals provide direction and focus for that motivation, helping an individual to channel their energy and efforts towards a specific objective. Also, people often confuse their desires with goals, but these are two distinct concepts. The method captures the desires and goals of the plurality of users, recognizes the difference between desire and goal, and helps the plurality of users develop a greater awareness of the role that these concepts play in motivation. The method also provides tools and techniques to help the plurality of users turn their desires into actionable goals. This can include techniques like affirmations to increase the intensity of the individual's desires and help them pursue their goals with greater determination and enthusiasm. Moreover, the purpose is the reason for which something is done or created or the intention behind an action or decision. Purpose can also refer to the sense of meaning or significance that an individual finds in their life or the goals and values that guide their actions. Purpose can be personal, in that it pertains to an individual's own motivations and aspirations, or it can be more collective, in that it relates to the aims and objectives of a group or organization. In general, purpose is what drives people to take action and helps to give their lives direction and meaning. The strength of a person's motivation is closely tied to the strength of their aspiration. Factors that can impact the strength of a person's aspiration include the clarity of their desire or goal, the frequency with which they think about it, and the emotional intensity that they feel around it. The method can use various ways to influence these factors and help individuals develop stronger aspirations and more powerful motivations. Furthermore, the strategies to create a purpose, desire, and goal for the plurality of users includes identifying the plurality of users values and goals, appeal to the plurality of users emotions, utilizing positive reinforcement, offering challenges and opportunities for growth, encouraging autonomy, fostering a supportive environment, considering strengths and skills, and reflecting on experiences and challenges.

FIG. 5C illustrates the list of attributes of the belief and mindset, according to an embodiment herein. the mindset, an internal motivation factor uses belief system, personal values, and personality types to model the baseline of the plurality of users and recommends the plurality of users perform a set of activities and tracks the progress. Based on the progress the method for personalized motivation management refines the recommendations. Furthermore, the method of measurement of mindset includes self-assessment questionnaires, data from other subsystems, and infrared data from the activities, engagements, and journals. In addition, the belief system helps to find the limiting beliefs which limit the plurality of users' ability and limit motivation level to achieve their goals. The method for measurement of the belief system includes self-assessment, task completion status, and inferred data from the plurality of user interactions.

FIG. 5D illustrates the list of attributes for the personal values and mindset, according to an embodiment herein. FIG. 5D illustrates the list of attributes of the personal values, which helps the plurality of users to identify the values and help them live by the values. In addition, the personality helps to provide the innate motivation elements of the plurality of users, and the method recommends the type of coaching based on personality types, data, and tendencies. Furthermore, mindset is a mental framework or set of assumptions that individuals use to interpret and make sense of the world around them. Key factors that can influence an individual's mindset include their belief system, values, and personality type. By understanding and managing these factors, individuals can learn to control their mindset and use it to manage their motivation and behavior.

FIG. 5E illustrates the list of attributes of the personality and mindset, according to an embodiment herein. FIG. 5E illustrates various attributes of the personality, which is a set of inherent traits and characteristics that shape the plurality of users behavior and motivations. There are various personality types, each with its own strengths and weaknesses. For example, individuals with a Type-A personality may be highly ambitious and driven, while those with a Type-B personality may be more relaxed and easy-going. Personality traits like conscientiousness, openness, and extraversion have been found to be positively related to intrinsic motivation, while traits like conscientiousness, extraversion, and neuroticism have been found to be positively related to extrinsic achievement motivation.

FIG. 5F illustrates the list of attributes of the energy system, according to an embodiment herein. FIG. 5F illustrates the energy state, which is the plurality of users perceived energy level, to achieve their goals and the energy state includes both physical energy and emotional energy of the plurality of users. Cultivating and sustaining heightened levels of energy will help a person to achieve their goals. Key sources of energy includes physical energy and emotional energy. Physical energy renew through sleep and rest, daytime rest, fitness and nutrition. Ways to manage physical energy includes healthy diet, hydration, exercise, sleep, and rest. Furthermore, the emotional energy includes the feelings of enjoyment, caring, satisfaction, and a sense of safety and trust. The emotional energy gets impacted positively and negatively, such as internal conflict due to moral issues, fairness issues, conflict issues, feeling empowered to set boundaries and focus, and sense of meaning and purpose in work.

FIG. 5G illustrates the list of attributes of the cognitive skills, according to an embodiment herein. FIG. 5G illustrates the cognitive skill of the plurality of users is assessed based on mindfulness, attention management, emotion regulation, and habit formation to manage motivation, the cognitive skill is tracked to improve the motivation skills of the plurality of users. Certain cognitive skills can significantly impact a person's motivation. These skills allow individuals to observe and potentially see more positive things, as well as regulate their emotions in a way that benefits their motivation. Some examples of cognitive skills that can influence motivation include mindfulness, attention management, emotion regulation, and habit formation. Mindfulness involves being self-aware of one's own mental state, emotions, and goals, as well as being aware of the present moment. This awareness can help to improve motivation by making goals more visible and attainable. For example, if you are mindful of your own energy levels, you can plan tasks that fit your current energy level and utilize your motivation to achieve them. When you are successful in achieving your goals, it can give you more confidence and increase your motivation. Additionally, mindfulness can help with awareness of distractions and the ability to regulate emotions in the face of distractions, which can improve motivation by allowing you to stay focused and on track. Overall, mindfulness can be a helpful tool in improving motivation and goal achievement. Furthermore, attention management is the ability to manage attention to the desired activities. Strategies include identifying distractions and eliminating them, creating a distraction-free environment, setting clear goals and priorities, using time-blocking and taking breaks. In addition, emotion regulation is the ability to regulate emotions, involves having knowledge and skills to manage and cope with a range of emotions. This capability can be especially important in maintaining motivation, as negative emotions can drain motivation and make it difficult to achieve goals. The techniques for regulating emotions, includes labeling and reframing, helps to manage motivation and stay on track towards your goals. For example, using stress management and emotional regulation techniques can help to reframe stressful situations in a more peaceful and manageable way, which can improve motivation. Overall, the ability to regulate emotions can be a valuable skill in maintaining motivation and achieve goals. Furthermore, habit formation is automatic actions that we perform without conscious thought. Forming positive habits can be a powerful way to improve motivation. For example, if you have established a habit of exercising in the morning or meditating regularly, these automatic actions can give you a boost in motivation. These habits can help to create a positive mental environment that supports and nourishes your motivation. Overall, establishing positive habits can be a helpful strategy in fostering and maintaining motivation.

FIG. 5H illustrates the list of attributes of the advancement, according to an embodiment herein. FIG. 5H illustrates the list of attributes of the advancement, which is the plurality of users feelings about the progress, they are making towards their goal and value. Personal growth or recognition at work or school is a recognition of their progress. When people feel they are progressing, they gain higher motivation. When the progress is made with fun, then this creates a much higher level of motivation. When the necessary job is part of the desire, this creates long-term motivation. Motivation can be sustained by gamifying (making it fun) for every task. Furthermore, the method offers gamification options to the plurality of users as advancement, an internal motivation factor. Hence, achievement can have a big impact on a person's motivation. When we achieve our goals, it can give us a sense of accomplishment and pride, which can in turn increase our motivation to continue pursuing our goals and aspirations. On the other hand, if we are unable to achieve our goals, it can lead to disappointment and a lack of motivation. Furthermore, achievement can have a positive impact on confidence. When we achieve our goals, which can boost our self-confidence. This increased confidence can then give us the motivation and determination to take on new challenges and continue pursuing our goals. In addition, the method keeps track of the progress and achievement the person is making—big or small and uses this periodically to boost motivation when the system notices the emotional energy level is low. The method also recommends people to formulate the goals for the desire and break down the goals into smaller chunks and have clear progress tracking on small parts. When the person sees they complete each small component, their confidence goes up in the expectation of the achievement which improves motivation.

FIG. 5I illustrates the list of attributes of the environment context, according to an embodiment herein. FIG. 5I illustrates the list of attributes of the environment context, which recommends strategies to change the context and measure the impact using the change in energy level. The context includes physical, social and informational. The physical context includes weather, food the user eats, and the place the user lives, the social context includes friends, family, and co-worker, and the informational context includes culture, policies, and process. Hence, environment context is one of the key motivational elements that influence the motivation of a person. This factor can be easily controlled. The physical environment context includes weather or climate, physically built environment—community resources like parks, nutrition environment, and neighborhood. The social environment context includes social support, norms, belief, and attitudes. Furthermore, the information environment includes policies, and processes, especially in the work environment.

FIG. 5J illustrates the list of attributes of the rewards and incentives, according to an embodiment herein. FIG. 5J illustrates the list of attributes of rewards and incentives. The method tracks the plurality of users rewards and incentives to accelerate the motivation level of the plurality of users. Further, the plurality of users uses the rewards and incentives to develop their own personal development system, which aligns with the goals to accelerate motivation. Rewards improve motivation in several ways, such as rewards provide a sense of accomplishment, rewards can increase intrinsic motivation, rewards can increase effort and rewards can increase engagement. Hence, it is important to consider the individual's goals and motivation style when using rewards to improve motivation, as different rewards may be more or less effective for different people. It is also important to use rewards appropriately, as overuse or misuse of rewards can lead to a decrease in motivation. The rewards can be of several types, such as verbal praise, physical rewards, social recognition, monetary awards, opportunities for advancement, and time off or flexible working hours or location.

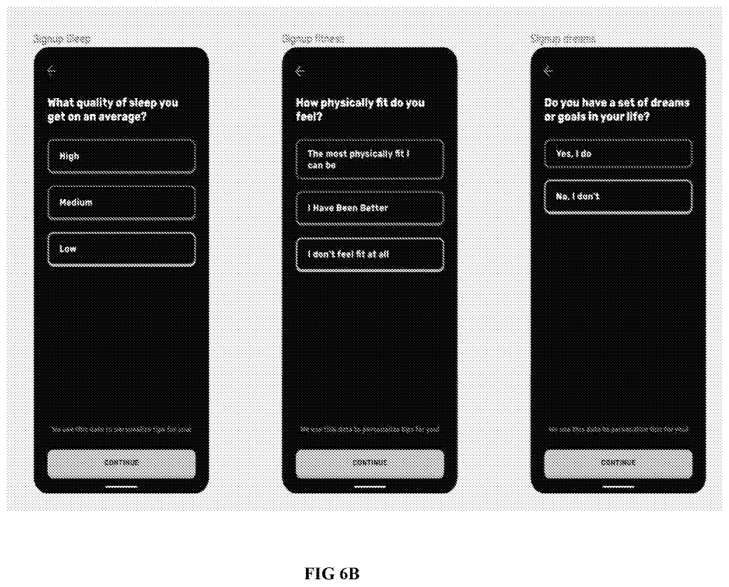

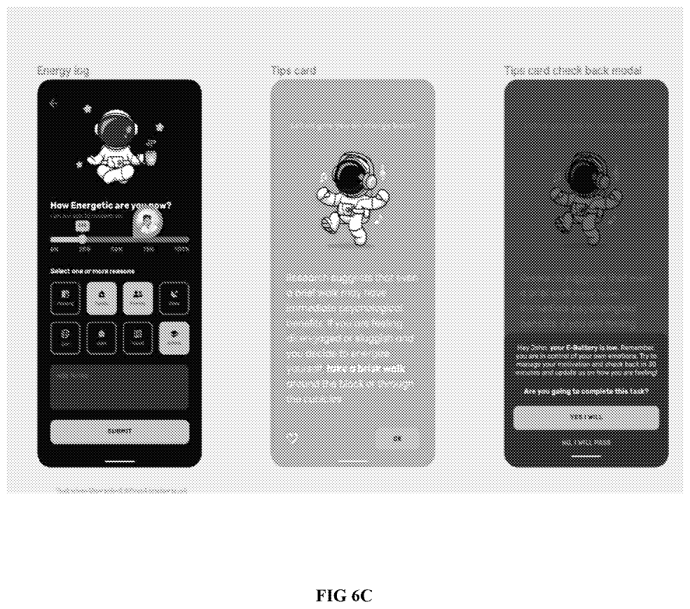

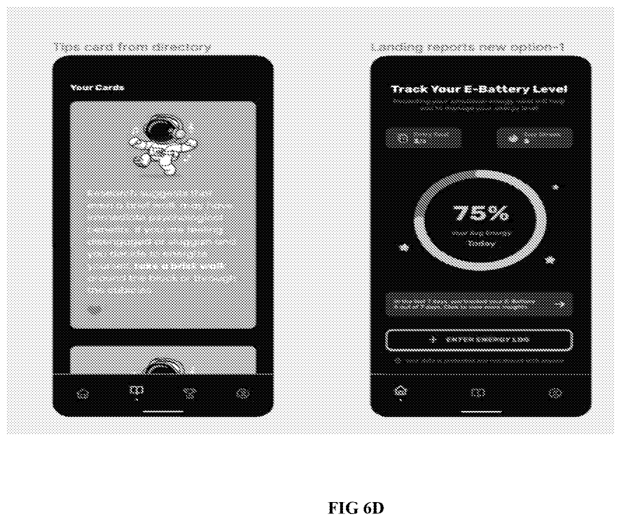

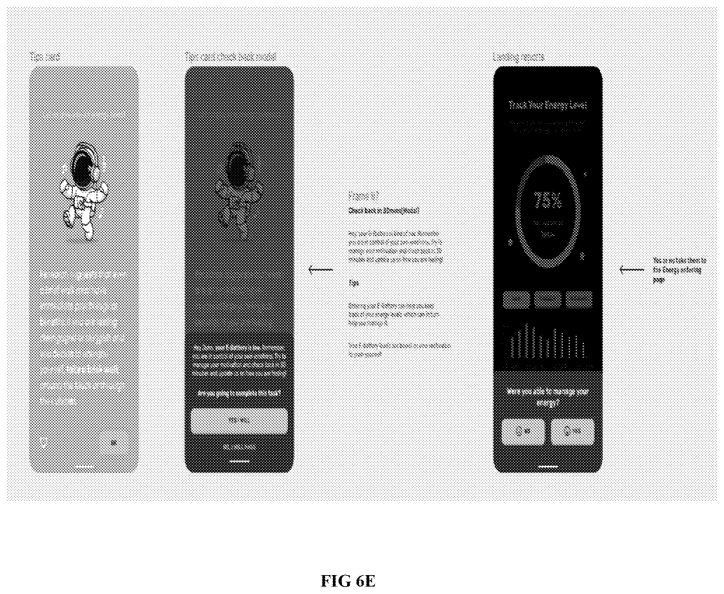

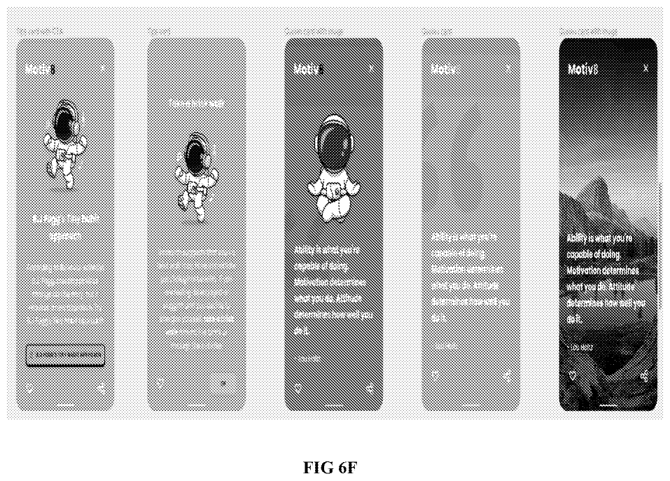

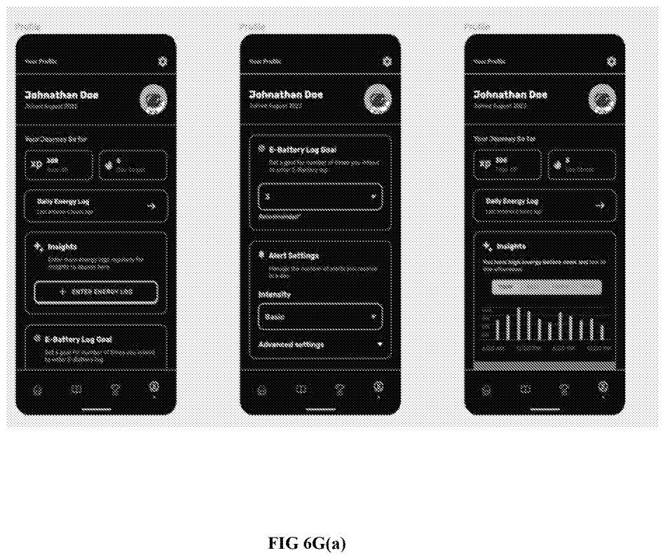

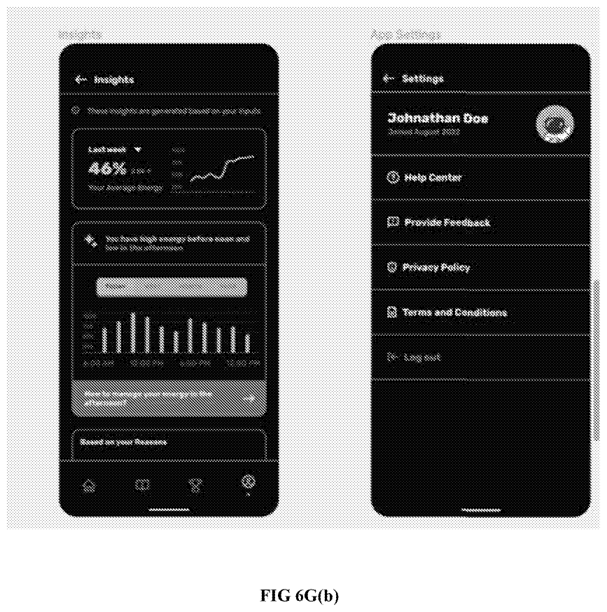

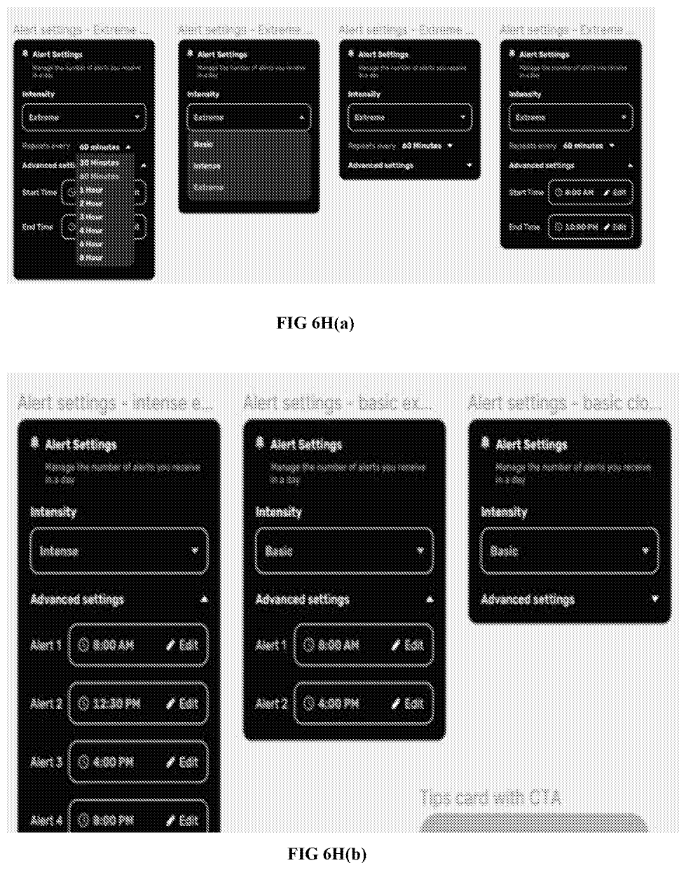

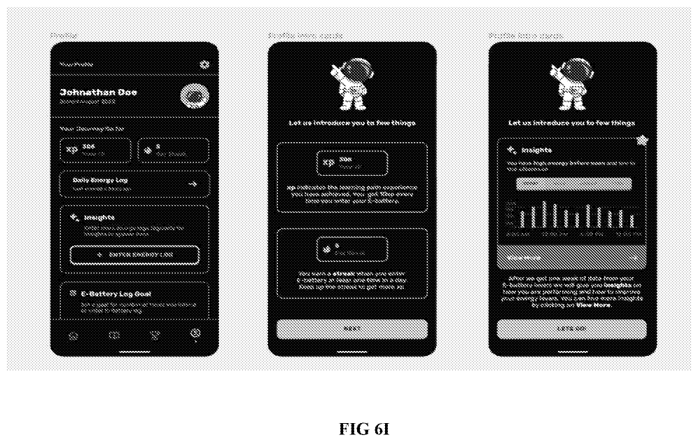

FIG. 6A-6I exemplarily illustrates screenshots of user interfaces personalized motivation management platform, according to an embodiment herein. FIG. 6A illustrates the screenshot of the signup user interfaces (UI). Signup UI with age, goal and time. FIG. 6B illustrates the screenshot of signup UI with sleep, fitness and dreams. FIG. 6C illustrates the screenshot of energy log, tip card and tip card check back model UI. FIG. 6D illustrates the screenshot of tip card and landing report on E-battery tracking level. FIG. 6E illustrates the screenshot of tip cards and landing reports UI. FIG. 6F illustrates the screenshot of tip card, quote card and quote card with image UI to improve the motivation level of the user. FIG. 6G illustrates the screenshot of profile of the user, insights and app settings UI. FIG. 6H illustrates the screenshot of various types of alert settings UI, such as extreme, intense and basic. FIG. 6I illustrates the screenshot of profile cards UI.

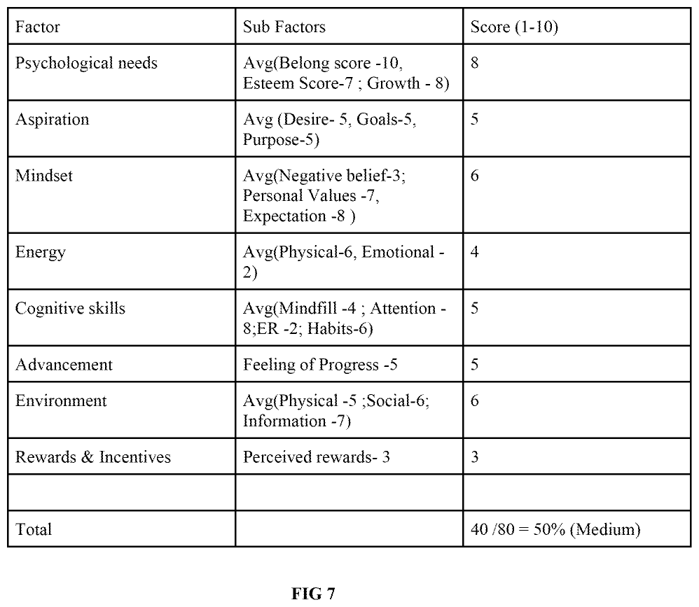

FIG. 7 illustrates a sample motivation score card generated for a user, according to an embodiment herein. FIG. 7 illustrates a sample motivation score card comprising plurality of motivation factors, such as psychological needs, aspiration, mindset, energy, cognitive skills, advancement, environment, rewards, and incentives. The score card also has motivation sub-factors and respective score from 1 to 10.

It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

While the disclosure is susceptible to various modifications and alternative forms, specific embodiment thereof has been shown by way of example in the drawings and will be described in detail above. It should be understood, however that it is not intended to limit the disclosure to the forms disclosed, but on the contrary, the disclosure is to cover all modifications, equivalents, and alternatives falling within the scope of the disclosure.

The embodiments herein disclose a computer-implemented system and method for personalized motivation management. The embodiments herein provides motivation management platform, that helps the individuals or organizations achieve their goals by actively managing and improving individual's motivation. This involves identifying and addressing factors that may be hindering motivation, such as a lack of energy to perform tasks or clear goals, lack of progress or achievement, or a negative mindset. The embodiments herein also involves providing individuals with the skills and resources they need to maintain and increase their motivation over time. The embodiments herein provide the individuals to manage their own motivation and achieve their goals. Also, helps to increase an individual motivation throughout the day. Further, the embodiments herein helps the companies to motivate their employees and improve satisfaction and productivity. The embodiments herein also helps the companies to analyze customer motivation and behavior and use insights to improve support and offer better products. Furthermore, life-coaches to help clients and teachers to help their students manage motivation and self-awareness to focus on the study and other goals.

Hence, the embodiments herein helps to improve productivity and well-being by helping individuals achieve their desired behavior or goals. By actively managing motivation, individuals may be more likely to persist in the face of challenges and setbacks, and to maintain a positive attitude and high level of engagement. This can lead to better outcomes and a more fulfilling and satisfying life.

Although the embodiments herein are described with various specific embodiments, it will be obvious for a person skilled in the art to practice the embodiments herein with modifications.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments.

It is to be understood that the phrases or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

What is claimed is:

1. A method (100) for personalized motivation management comprising steps of:
a. extracting a plurality of motivation attributes from a plurality of users, through a plurality of external applications, websites, and external devices connected with the plurality of users, and additionally collecting external data through an application programming interface (API) (101); and wherein the plurality of motivation attributes includes the user's current motivation level, behavior, skill level, and desired goals;
b. dynamically generating one or more motivation models for each of the plurality of users based on a plurality of motivation modeling criteria derived from a plurality of motivation factors (102); and wherein the plurality of motivation factors comprises a plurality of internal motivation factors and a plurality of external motivation factors; and wherein the plurality of internal motivation factors includes psychological needs, aspirations, mindset, advancement, cognitive skills, and energy state; and wherein the plurality of external motivation factors includes environment context, rewards and incentives;
c. generating a motivation score for each of the plurality of users by measuring the plurality of user's motivation levels, and scoring the plurality of motivation factors and associated plurality of motivation sub-factors (103); and wherein the plurality of user's motivation is measured through a plurality of self-assessment questions, system calculation, and user activity data;
d. dynamically generating one or more targeted, control elements specific to a target user, among the plurality of users using the one or more motivation models, the motivation score, and one or more content libraries (104); and wherein the one or more targeted, control elements include the plurality of motivation factors influencing the plurality of users behavior and help in achieving the motivation goal; and
e. rendering the one or more targeted, control elements on a user device of the target user through a plurality of delivery channels (105).

2. The method (100) according to claim 1, wherein the plurality of motivation attributes from the plurality of users is also extracted through a questionnaire, and wherein the plurality of external applications include chatbot, mobile applications, web interface, and/or web portal; and wherein the plurality of external devices include smart watches, smartphones, computers, and/or laptops.

3. The method (100) according to claim 1, wherein the one or more motivation models is a digital twin of the plurality of users, and which represents the plurality of users' motivation level, and wherein the plurality of users is enabled to recognize the user's motivation level at a given moment and maintain a motivation level required to accomplish the plurality of users goal.

4. The method (100) according to claim 1, wherein the one or more motivation models generated serves as a model of a desired future condition or status by providing visibility and insights into a present condition or status, by offering a plurality of recommendations for a future condition or status, and wherein the plurality of recommendations for the future condition or status comprises a range of knowledge content and skill-building activities for the plurality of user for achieving the goals, and wherein the plurality of recommendations for the future condition or status is delivered directly to the plurality of users as a micro-content through a plurality of applications or through a plurality of channels including WhatsApp, slack, and/or email, and wherein the pluralityof skill building activities include competitions, affirmations, journaling, and games; and wherein the plurality of recommendations include a live assistance and recommendations from a plurality of personal coaches or artificial intelligence (AI) based chatbots, to help the plurality of users to practice concepts in real life, and wherein the plurality of tools include energy management tools, focus timers, goal-tracking tools and progress tracking tools.

5. The method (100) according to claim 1, further comprises monitoring a progress of the plurality of users and wherein the plurality of recommendations provided are modified in real time based on an output result obtained due to the plurality of previously provided recommendations.

6. The method (100) according to claim 1, wherein the one or more motivation models help to understand the various factors that influence the plurality of users' motivation and to use this understanding to increase performance efficiency, productivity, and overall well-being, and wherein the one or more motivation models are also employed to provide a framework for predicting and adjusting the motivation levels, and wherein the one or more motivation models are also employed to identify a plurality of strategies or interventions used to either increase or decrease a motivation in different situations, and wherein the one or more motivation models are further employed to understand and increase motivation in order to increase the output efficiency.

7. The method (100) according to claim 1, wherein the psychological needs include a feeling of belonging, esteem needs, and growth; and wherein the feeling of belonging includes acceptance of social groups small or large, need for family, and friends, intimacy, trust, acceptance, receiving and giving love and affection, and wherein the esteem needs includes self-respect and respect from others, need for status, recognition, fame, prestige, and attention; and wherein the growth includes competence, education, mastery, self-confidence, autonomy, and freedom; and wherein the psychological needs are captured using self-assessment questionnaires and the score gets refined over time based on the insights from the plurality of user's interaction; and wherein a method of measurement of psychological needs include self-assessment, task completion status, inferred data from the plurality of user interactions.

8. The method (100) according to claim 1, wherein an aspiration and an internal motivation factor are responsible for identifying and tracking a plurality of desires or requirements, goals, and purposes of the plurality of users; and the aspiration is used to manage and maintain the motivation of the plurality of users; and wherein a method of measurement of aspiration include self-assessment, task completion status, inferred data from the plurality of user interactions.

9. The method (100) according to claim 1, wherein a mindset or attitude, an internal motivation factor uses belief system, personal values, and personality types to model a baseline of the plurality of users and recommends the plurality of users to perform a set of activities and tracks the progress; and wherein the recommendations are refined or fine-tuned or modified based on a progress or improvement or increase in work efficiency; and wherein a method of measurement of mindset includes self-assessment of questionnaires, data from other subsystems, and infrared data from the activities, engagements, and journals.

10. The method (100) according to claim 9, wherein the belief system helps to find a limiting beliefs which limit the plurality of users ability and limit motivation level to achieve their goals; and wherein a method of measurement of belief system includes self-assessment, task completion status, inferred data from the plurality of user interactions; and wherein the personal values helps the plurality of users to identify the values and help them live by the values; and wherein the personality helps to provide the innate motivation elements of the plurality of users; and wherein a type of coaching recommended is selected based on a personality type, data, and tendency.

11. The method (100) according to claim 1, wherein the energy state is the plurality of users perceived energy level, to achieve their goals; and wherein the energy state includes both physical energy and emotional energy of the plurality of users; and wherein the cognitive skill of the plurality of users is assessed based on mindfulness, attention management, emotion regulation, and habit formation to manage motivation; and wherein the cognitive skill is tracked to increase the motivation skills of the plurality of users.

12. The method (100) according to claim 1, wherein the advancement is the plurality of users feelings about the progress, made by them towards their goals and values; and wherein a plurality of gamification options is provided to the plurality of users as advancement, and an internal motivation factor; and wherein the environment context recommends strategies to change the context and measure an impact using the change in energy level; and wherein the context includes physical, social and informational contexts; and wherein the physical context includes weather, food eaten by the user, and a place in which the user lives; and wherein the social context includes friends, family, and co-workers; and wherein the informational context includes culture, policies, and process.

13. The method (100) according to claim 1, further comprises tracking the plurality of users rewards and incentives to accelerate the motivation level of the plurality of users; and wherein the plurality of users uses the rewards and incentives to develop their own personal development system, that align with the goals to accelerate motivation.

14. The method (100) according to claim 1, wherein the process step for managing the one or more motivation models comprises:
a. creating a motivation baseline for the plurality of users through questionnaire, skill assessment, activities and response to activities;
b. creating an improvement plan based on the motivation baseline;
c. gamifying the improvement plan for each of the plurality of users;
d. engaging with the plurality of users through a game; and wherein for an individual user the game is a single-player game; and wherein for a company the game is a multi-player game with a plurality of employees or a single-player game, when an individual employee is involved;
e. measuring continuously and refining strategies for uplifting motivation;
f. providing insights to the plurality of users on the current state and progress;
g. learning from the similar plurality of users and incorporating learning back to the general population;
h. managing motivation level across various group and taking strategic action within a company; and managing individually using a single application for each of the plurality of users;
i. personalizing various levels including individual level, corporate level or based on a coach's input;
j. understanding the concepts based on knowledge content; and wherein the knowledge content is a content repository comprising images, tiny tips card, letters, emails, chat messages, videos and presentations; and
k. using a combination of coach and an individual user as a multi-user usage.

15. The method (100) according to claim 1, wherein the self-assessment questions includes input provided by the plurality of user as a set of questions; and wherein the user activity data is collected through using an application, website, or third-party applications providing insight into the plurality of users' motivation and related factors.

16. The method (100) according to claim 1, wherein the motivation score is generated based on the plurality of motivation factors and sub-factors; and wherein the motivation score is the product of individual factor score and weightage of the factor; and wherein the weightage of the factor is in the range of 0 to 10; and wherein the motivation score is categorized as low (<25% score), medium (>25% and <75%), and high (>75%).

17. The method (100) according to claim 14, wherein the improvement plan includes providing tiny tips, comprising small pieces of educational content to improve the lowest score of the plurality of users; and wherein the engaging the plurality of users includes through a just-in-time recommendation or a tip through a card as the plurality of users enter data, displaying motivating images or quotes to uplift the plurality of users, offering options to the plurality of users to personalize the applications and the content the plurality of users viewing, using in-app alerts to share pieces of content or insights, sharing content with others by the plurality of users, sending small tips to the plurality of users through messaging applications including WhatsApp, slack, and Microsoft® Teams, using an interactive bot to provide insights to the plurality of users and collecting input and feedback.

18. The method (100) according to claim 14, wherein the learning from the similar plurality of users includes best practices based on the user personas and patterns; and wherein the best practices are given more weightage and recommendations across the similar plurality of users; and wherein the personalizing various levels includes customizing and setting preferences for the tip cards, images and messages entered by the plurality of users to improve their mood.

19. The method (100) according to claim 1, wherein the user device comprises a smartwatch, smartphone, computer, and/or laptop; and wherein the plurality of delivery channels include smartwatches, smartphones, applications in smartwatches and smartphones, bots including Microsoft® Teams, and web interfaces.

20. A computer-implemented system (200) for personalized motivation management comprising:
a. an attribute extraction module (201) configured to extract a plurality of motivation attributes from a plurality of users through external applications, websites, and external devices the plurality of users interacts with, and additionally collecting external data through an application programming interface; and wherein the plurality of motivation attributes includes the user's current motivation level, behavior, skill level, and desired goals;
b. a motivation modelling module (202) configured to generate one or more motivation models for each of the plurality of users based on motivation modeling criteria derived from a plurality of motivation factors; and wherein the plurality of motivation factors comprises internal motivation factors and external motivation factors; and wherein the internal motivation factors include psychological needs, aspirations, mindset, advancement, cognitive skills, and energy state; and wherein the external motivation factors include environment context, rewards and incentives;
c. a motivation score generating module (203) configured to generate a motivation score for each of the plurality of users by measuring the plurality of user's motivation, and scoring the plurality of motivation factors and associated plurality of motivation sub-factors; and wherein the plurality of user's motivation is measured through self-assessment questions, system calculation, and user activity data;
d. a motivation management subsystem module (205) configured to dynamically generate one or more targeted, control elements specific to a target user and render the one or more targeted, control elements on a user device of the target user through a plurality of delivery channels, among the plurality of users using the one or more motivation models generated by the motivation modelling module, the motivation score generated by the motivation score generating module, and one or more content libraries; and wherein the one or more targeted, control elements include the plurality of motivation factors, influencing the plurality of users behavior and help in achieving the motivation goal.

21. The system (200) according to claim 20, wherein the attribute extraction module is also configured to extract plurality of motivation attributes from the plurality of users through a questionnaire; and wherein the external applications include chatbot, mobile applications, web interface, and/or web portal; and wherein the external devices include smart watches, smartphones, computers, and/or laptops.

22. The system (200) according to claim 20, wherein the motivation modelling module configured to generate one or more motivation models is a digital twin of the plurality of users, which represents the plurality of users' motivation level; and wherein the system (200) further assists the plurality of users in recognizing the user's motivation level at that moment and maintain the motivation necessary to accomplish the plurality of users' goal.

23. The system (200) according to claim 20, wherein the motivation modelling module configured to generate one or more motivation models serves as a model of the desired future state by providing visibility and insights into the current state, and by offering recommendations through a recommendation module (206) for the future state; and wherein the recommendation module (206) in conjunction with the motivation modelling module (202) configured to generate recommendations for the future state comprises range of knowledge content and skill-building activities for the plurality of users to achieving their goals; and wherein the recommendations for the future state are delivered to the plurality of users as a micro-content delivered directly through the applications or through channels including WhatsApp, slack, and/or email; through engaging activities including competitions, affirmations, journaling, and games; through live assistance and recommendations from real personal coaches or artificial intelligence (AI) based chatbots; and as a tool to help the plurality of users to practice concepts in real life including energy management tools, focus timers, goal-tracking tools and progress tracking tools.

24. The system (200) according to claim 20, further monitors the progress of the plurality of users and the recommendations provided by the recommendation module (206) in conjunction with the motivation modelling module (202) is adjusted in real-time based on the effectiveness of the previous recommendations.

25. The system (200) according to claim 20, wherein the motivation modelling module (202) configured to generate the one or more motivation models help to understand the various factors that influence the plurality of users' motivation and to use this understanding to improve performance, productivity, and overall well-being; and wherein the one or more motivation models also aim to provide a framework for predicting and adjusting the motivation levels; and wherein the one or more motivation models also identifies strategies or interventions used to either increase or decrease motivation in different situations; and wherein the one or more motivation models further helps to understand and enhance motivation in order to improve the outcomes.

26. The system (200) according to claim 20, wherein the psychological needs include the feeling of belonging, esteem needs, and growth; and wherein the feeling of belonging includes acceptance of social groups small or large, need for family, and friends, intimacy, trust, acceptance, receiving and giving love and affection, and wherein the esteem needs includes self-respect and respect from others, need for status, recognition, fame, prestige, and attention; and wherein the growth includes competence, education, mastery, self-confidence, autonomy, and freedom; and wherein the psychological needs are captured using self-assessment questionnaires and the score gets refined over time based on the insights from the plurality of user's interaction; and wherein the method of measurement of psychological needs include self-assessment, task completion status, inferred data from the plurality of user interactions.

27. The system (200) according to claim 20, wherein the aspiration, an internal motivation factor is responsible for identifying and tracking the various desires, goals, and purposes of the plurality of users; and wherein the system (200) utilizes aspiration to manage and maintain the plurality of users motivation; and wherein the method of measurement of aspiration include self-assessment, task completion status, inferred data from the plurality of user interactions.

28. The system (100) according to claim 20, wherein the mindset, an internal motivation factor uses belief system, personal values, and personality types to model the baseline of the plurality of users and recommends the plurality of users, to perform a set of activities and tracks the progress; and wherein based on the progress, the system (200) refines the recommendations; and wherein the method of measurement of mindset includes self-assessment questionnaires, data from other subsystems, and infrared data from the activities, engagements, and journals.

29. The system (200) according to claim 28, wherein the belief system helps to find the limiting beliefs which limit the plurality of users ability and limit motivation level to achieve their goals; and wherein the method of measurement of belief system include self-assessment, task completion status, inferred data from the plurality of user interactions; and wherein the personal values helps the plurality of users to identify the values and help them live by the values; and wherein the personality helps to provide the innate motivation elements of the plurality of users; and wherein the system (200) further recommends the type of coaching based on the personality types, data, and tendencies.

30. The system (200) according to claim 20, wherein the energy state is the plurality of users perceived energy level, to achieve their goals; and wherein the energy state includes both physical energy and emotional energy of the plurality of users; and wherein the cognitive skill of the plurality of users is assessed based on mindfulness, attention management, emotion regulation, and habit formation to manage motivation; and wherein the cognitive skill is tracked to improve the motivation skills of the plurality of users.

31. The system (200) according to claim 20, wherein the advancement is the plurality of users feelings about the progress, they are making towards their goal and value; and wherein the system (200) also offers gamification options to the plurality of users as advancement, an internal motivation factor; and wherein the environment context recommends strategies to change the context and measure the impact using the change in energy level; and wherein the context includes physical, social and informational; and wherein the physical context includes weather, food the user eats, and the place the user lives; and wherein the social context includes friends, family, and co-workers; and wherein the informational context includes culture, policies, and process.

32. The system (200) according to claim 20, further tracks the plurality of users rewards and incentives to accelerate the motivation level of the plurality of users; and wherein the plurality of users uses the rewards and incentives to develop their own personal development system, that aligns with the goals to accelerate motivation.

33. The system (200) according to claim 20, wherein the motivation modelling module configured to generate one or more motivation models are managed by the method comprising the steps of:
a. creating a motivation baseline for the plurality of users through questionnaire, skill assessment, activities and response to activities;
b. creating an improvement plan based on the motivation baseline;
c. gamifying the improvement plan for each of the plurality of users;
d. engaging with the plurality of users through a game; and wherein for an individual user the game is a single-player game; and wherein for a company the game is a multi-player game with a plurality of employees or a single-player game, when an individual employee is involved;
e. measuring continuously and refining strategies for uplifting motivation;
f. providing insights to the plurality of users on the current state and progress;
g. learning from the similar plurality of users and incorporating learning back to the general population;
h. managing motivation level across various group and taking strategic action within a company; and managing individually using a single application for each of the plurality of users;
i. personalizing various levels including individual level, corporate level or based on a coach's input;
j. understanding the concepts based on knowledge content; and wherein the knowledge content is a content repository comprising images, tiny tip cards, letters, emails, chat messages, videos and presentations; and
k. using a combination of coach and an individual user as a multi-user usage.

34. The system (200) according to claim 20, wherein the self-assessment questions includes input provided by the plurality of user as a set of questions; and wherein the user activity data is collected through using an application, website, or third-party applications providing insight into the plurality of users' motivation and related factors.

35. The system (200) according to claim 20, wherein the motivation score generating module configured to generate motivation score is generated based on the plurality of motivation factors and sub-factors; and wherein the motivation score is the product of individual factor score and weightage of the factor; and wherein the weightage of the factor is in the range of 0 to 10; and wherein the motivation score is categorized as low (<25% score), medium (>25% and <75%), and high (>75%).

36. The system (200) according to claim 33, wherein the improvement plan includes providing tiny tips, comprising small pieces of educational content to improve the lowest score of the plurality of users; and wherein the engaging the plurality of users includes through a just-in-time recommendation or a tip through a card as the plurality of users enter data, displaying motivating images or quotes to uplift the plurality of users, offering options to the plurality of users to personalize the applications and the content the plurality of users viewing, using in-app alerts to share pieces of content or insights, sharing content with others by the plurality of users, sending small tips to the plurality of users through messaging applications including WhatsApp, slack, and Microsoft® Teams, using an interactive bot to provide insights to the plurality of users and collecting input and feedback.

37. The system (200) according to claim 33, wherein the learning from the similar plurality of users includes best practices based on the user personas and patterns; and wherein the best practices are given more weightage and recommendations across the similar plurality of users; and wherein the personalizing various levels includes customizing and setting preferences for the tip cards, images and messages entered by the plurality of users to improve their mood.

38. The system (200) according to claim 20, wherein the user device comprises a smartwatch, smartphone, computer, and/or laptop; and wherein the plurality of delivery channels include smartwatches, smartphones, applications in smartwatches and smartphones, bots including Microsoft® Teams, and web interfaces.
